(19) United States
(12) Patent Application Publication
(10) Pub. No.: US 2002/0168652 A1
(43) Pub. Date: Nov. 14, 2002

(54) Surface assembly for immobilizing DNA capture probes and bead-based assay including optical bio-discs and methods relating thereto

(76) Inventors: Martina Elisabeth Werner, Aliso Viejo, CA (US); Ramon Magpantay Valencia, Aliso Viejo, CA (US); Jorma Antero Virtanen, Las Vegas, NV (US); Jim Vincent Zoval, Lake Forest, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/035,836

(22) Filed: Dec. 21, 2001

Related U.S. Application Data
(60) Provisional application No. 60/257,705, filed on Dec. 22, 2000.
(60) Provisional application No. 60/259,806, filed on Jan. 4, 2001.
(60) Provisional application No. 60/292,110, filed on May 18, 2001.
(60) Provisional application No. 60/313,917, filed on Aug. 21, 2001.

(51) Int. Cl. C12Q 1/68; G06F 19/00; G01N 33/48; G01N 33/50; C12M 1/34

(52) U.S. Cl. 435/6; 435/287.2; 702/20

(57) ABSTRACT

An optical bio-disc that may be used to test a sample for a target nucleic acid of a prescribed sequence is described. The bio-disc includes a flow channel having target or capture zones, a return channel in fluid communication therewith, and in some embodiments a mixing chamber in fluid communication with the flow channel. The bio-disc may be implemented on an optical disc including an information encoding format such as CD, CD-R, or DVD or a modified version thereof. Methods of manufacturing the optical bio-disc and methods of using the optical bio-disc to detect nucleic acids according to the present invention are also provided.

### CROSS-REFERENCE TO RELATED APPLICATIONS

This application claims the benefit of U.S. Provisional Applications, Ser. Nos. 60/257,705, filed Dec. 22, 2000; 60/259,806, filed Jan. 4, 2001; 60/292,110, filed May 18, 2001; and 60/313,917, filed Aug. 21, 2001; the contents of all of which are hereby incorporated by reference in their entireties.

### BACKGROUND OF THE INVENTION

The present invention relates to methods and systems for the detection of nucleic acids, including deoxyribonucleic acids (DNA) and ribonucleic acids (RNA). It relates more particularly to a bead based assay system utilizing reflective and/or transmissive optical discs for detection of nucleic acids.

Assay systems utilizing optical discs have been described. See, for example, Virtanen, U.S. Pat. No. 6,030,581, entitled Laboratory in a Disk. Such systems have enormous potential in the field of medicine, for diagnostic and other clinical assays, as well as in fields such as environmental testing and the like. Nonetheless, there remains a continuing need to develop assays that are faster, more efficient, and more economical.

Assays that detect the presence of specific sequences of nucleic acids have a number of applications. For example, nucleic acid detection systems are used to test for the presence of specific disease causing agents, such as viruses or bacteria, in biological samples taken from patients. Nucleic acid detection systems are also used to test water and soil samples for specific microorganisms. Indeed, nucleic acid testing can be used to identify particular strains or types of a microorganism, which may have important implications for the appropriate response or treatment. Nucleic acid testing is also helpful in monitoring agricultural products as, for example, in testing for the presence of genetically modified crop products. As is well known, nucleic acid testing has important forensic applications as well.

What is needed, therefore, is a rapid, efficient, and economical assay system for testing various samples for specific nucleic acid sequences.

### SUMMARY OF THE INVENTION

This invention relates to identification of a target DNA or RNA that may exist in a sample and test methods relating thereto. The invention is further directed to an optical bio-disc used to test a sample of DNA or RNA for a target DNA or RNA of a prescribed sequence. The bio-disc includes a flow channel having target or capture zones, a return channel in fluid communication therewith, and in some embodiments a mixing chamber in fluid communication with the flow channel. The bio-disc may be implemented on an optical disc including an information encoding format such as CD, CD-R, or DVD or a modified version thereof. Methods of manufacturing the optical bio-disc according to the present invention are also provided.

A bio-disc drive assembly is employed to rotate the disc, read and process any encoded information stored on the disc, and analyze the DNA samples in the flow channel of the bio-disc. The bio-disc drive is thus provided with a motor for rotating the bio-disc, a controller for controlling the rate of rotation of the disc, a processor for processing return signals form the disc, and an analyzer for analyzing the processed signals. The rotation rate is variable and may be closely controlled both as to speed, direction, and time of rotation. The bio-disc drive assembly may also be utilized to write information to the bio-disc either before, during, or after the test material in the flow channel and target zones is interrogated by the read beam of the drive and analyzed by the analyzer. The bio-disc may include encoded information for controlling the rotation rate of the disc, providing processing information specific to the type of DNA or RNA test to be conducted, for displaying the results on a monitor associated with the bio-drive, and/or saving the results on a hard drive, floppy disc, on the bio-disc itself, or on any other recordable media. A reflective disc format suitable for use in the present invention is disclosed in commonly assigned U.S. Provisional Application 60/249,391 entitled “Optical Disc Assembly for Performing Microscopy and Spectroscopy Using Optical Disc Drive,” hereby incorporated by reference in its entirety.

In an alternative embodiment, a transmissive disc format may be used in which the interrogation beam is transmitted through the target zone and detected by a top detector. Such a transmissive disc format is disclosed in commonly assigned U.S. Pat. No. 6,327,013 and in commonly assigned U.S. Provisional Applications Nos. 60/293,917; 60/303,437; and 60/323,405, entitled “Optical Discs and Assemblies for Detection of Microscopic Structures Using Focal Zone Control,” hereby incorporated by reference in their entireties.

Development of a DNA based assay for CD, CD-R, or DVD formats and variations thereof according to the present invention, includes attachment of micro particles or beads to the disc surface as a detection method. These particles or beads are selected in size so that the read or interrogation beam of the drive can “see” or detect a change of surface reflectivity or transmittance caused by the particles.

The beads are bound to the disc surface through DNA hybridization. A capture probe is attached to the disc, while a signal probe is attached to the beads. Each of these probes are complementary to a different portion of the target sequence, but are not complementary to each other. In the presence of a target sequence, both capture and signal probes hybridize with the target. In this manner, the beads are attached to a disc surface. In a subsequent centrifugation (or wash) step, all unbound beads are removed. Alternatively, the target itself is directly bound or linked to the beads without the presence of an extra signaling probe.

A number of different surface chemistries and different methods for binding the capture probes to the disc surface were investigated. One observed result was unspecific binding of beads to the relevant disc surface. This limitation was overcome by the development of a method for attaching the capture probes to the disc surface by use of an active layer which has no or negligible unspecific affinity for the reporters according to the present invention. The active layer is also utilized to anchor the capture DNA through interaction with a reactive group present on the capture DNA including, but not limited to, amino, thiol, carboxyl, aldehyde, and hydroxyl groups. Specific bead binding is achieved through DNA hybridization. Thus in a preferred embodiment of the present invention, the capture probes are connected to an amino (NH₂) group, and the disc surface is coated with a layer of modified polystyrene, preferably polystyrene-co-maleic anhydride. The NH₂ group binds (covalently) to the maleic anhydride, thereby attaching the DNA capture probe to the disc surface in a target or capture zone. Alternatively, the active layer may be formed from gold, activated glass, modified glass, or other modified media. The modified media includes anhydride groups, activated carboxylate groups, or carboxylic acid aldehyde groups.

Subsequently, DNA hybridization and bead binding occurs. After separating the unbound beads, specific binding of beads can be detected using different methods. These methods include microscopic analysis, measurement of fluorescence signal on the disc surface using a FluorImager (Molecular Dynamics), or bead detection in a CD-type reader. Event counting software useful for bead detection in a CD-type reader is disclosed in commonly assigned U.S. Provisional Application 60/291,233 entitled “Variable Sampling Control for Rendering Pixelization of Analysis Results in Optical Bio-disc Assembly and Apparatus Relating Thereto,” hereby incorporated by reference in its entirety.

The DNA assay according to the present invention may be implemented in an open disc format as well as in a micro channel. In the open disc format, the reagents are spotted directly on the disc surface. Unbound reagents are removed by washing the disc. In the micro channel format, the DNA binding is initially performed on an open disc substrate. After attaching the DNA capture probes, the channel is assembled by affixing adhesive and a cover disc or cap. Subsequent steps are performed in the closed channel which is filled with liquids such as buffer solutions, bead suspensions, and DNA test samples which are analyzed for the presence of a target sequence. BRIEF DESCRIPTION OF THE DRAWINGS

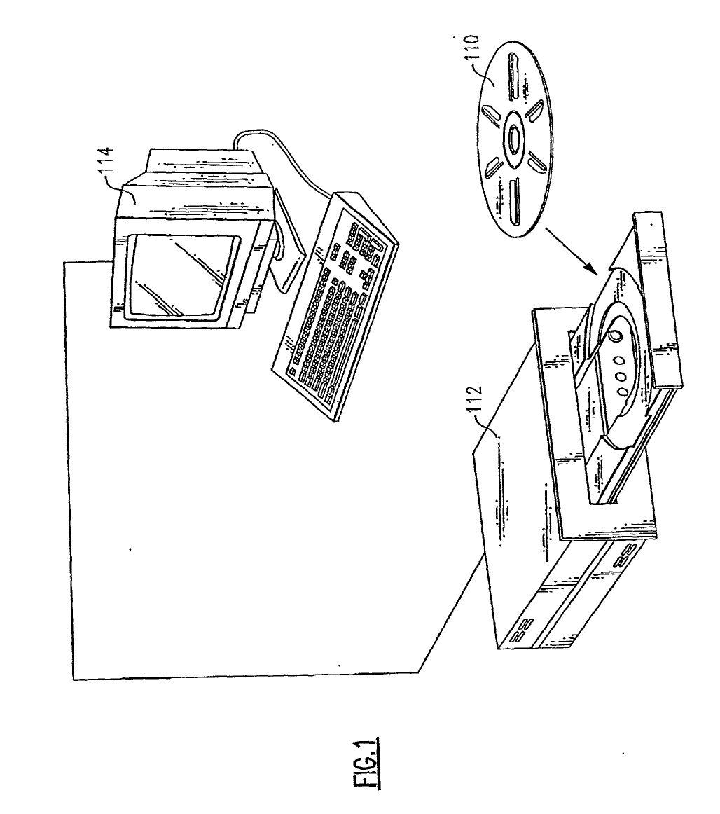

FIG. 1 is a pictorial representation of a bio-disc system according to the present invention;

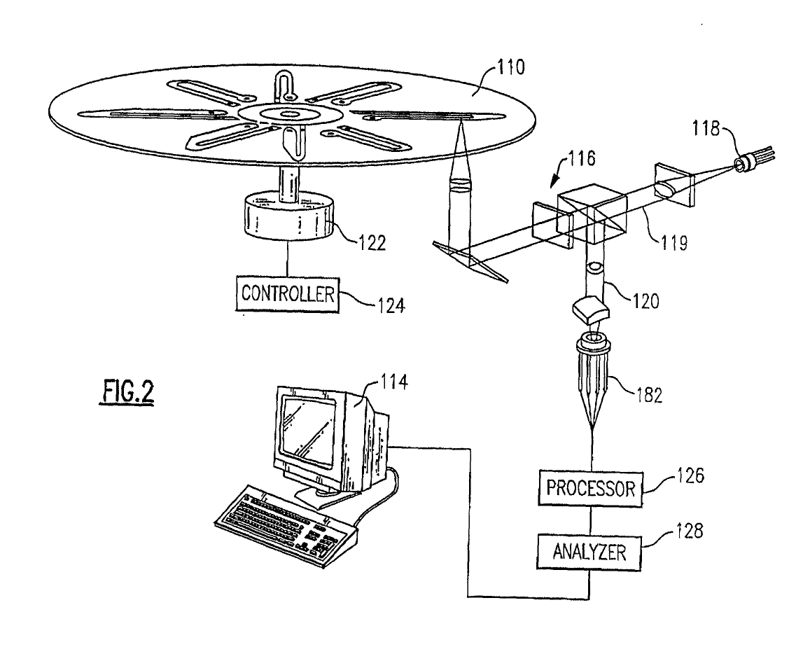

FIG. 2 is a perspective and block diagram representation illustrating the system of FIG. 1 in more detail;

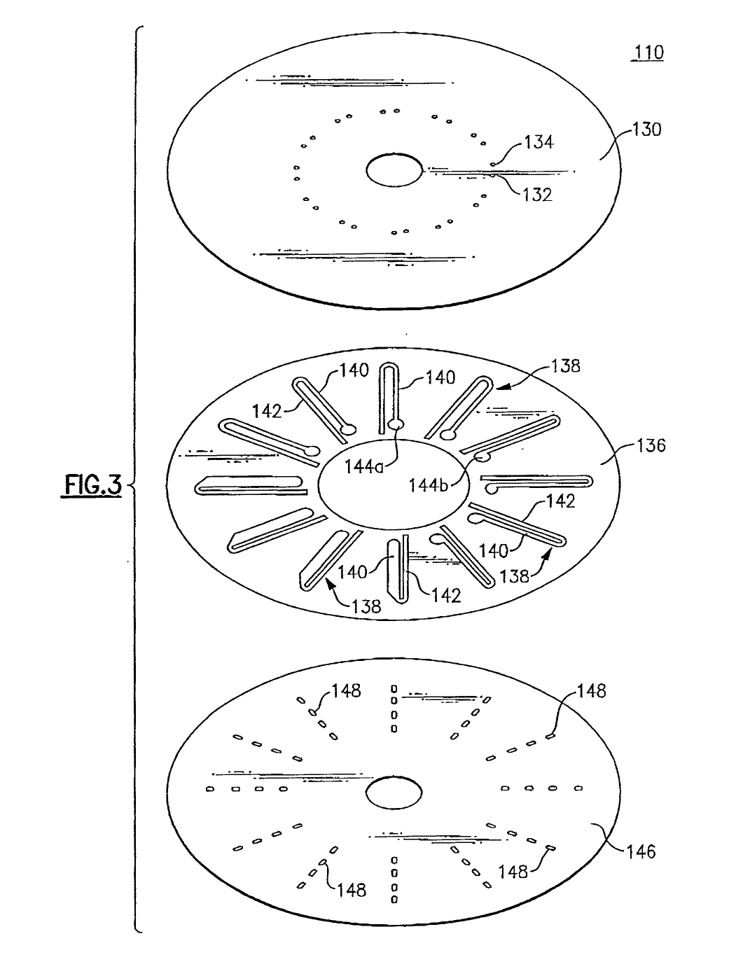

FIG. 3 is an exploded perspective view of a reflective bio-disc as utilized in conjunction with the present invention;

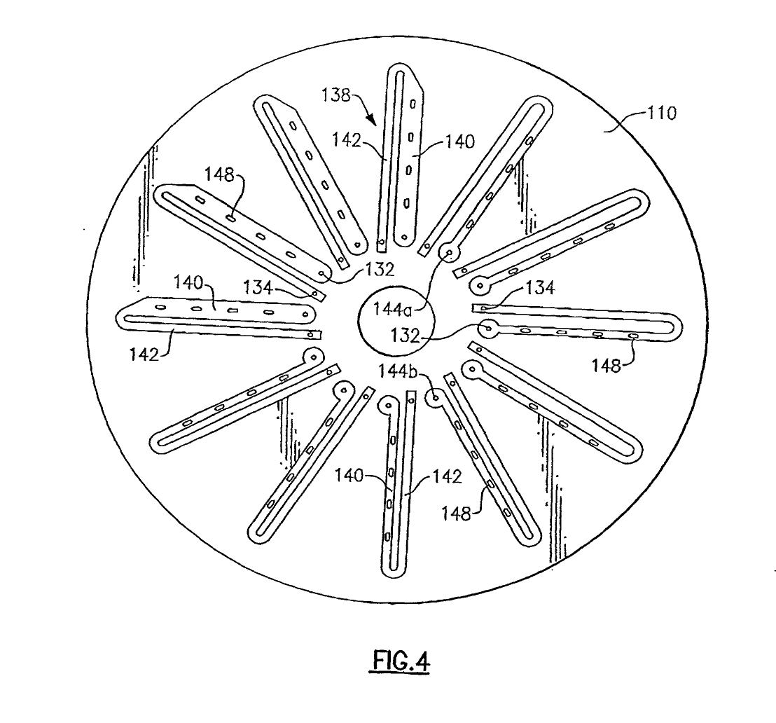

FIG. 4 is a top plan view of the disc shown in FIG. 3;

FIG. 5 is a perspective view of the disc illustrated in FIG. 3 with cut-away sections showing the different layers of the disc;

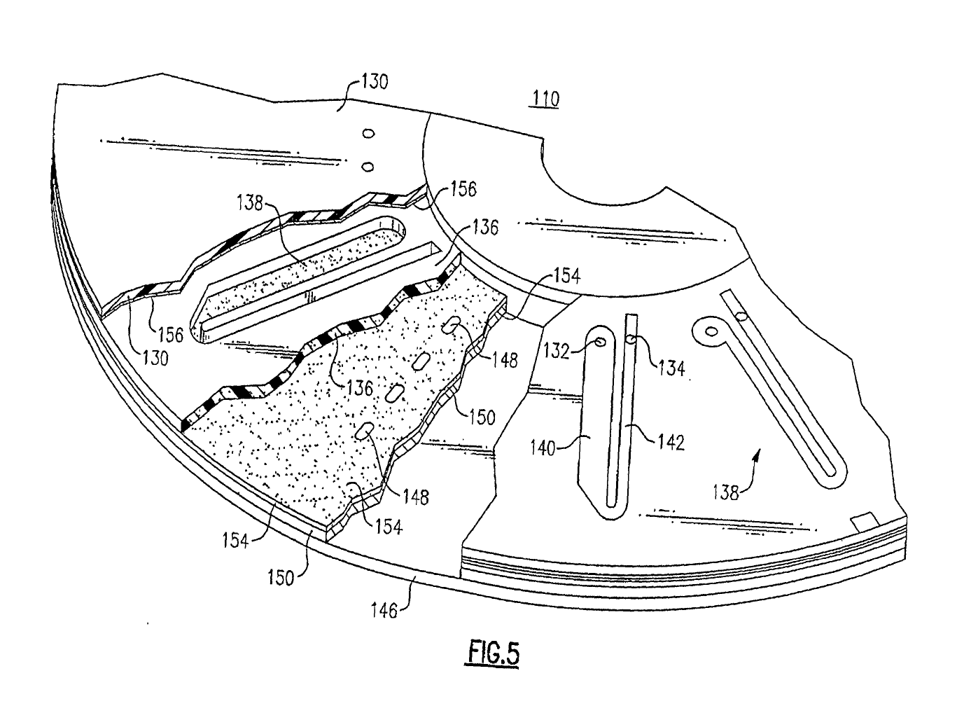

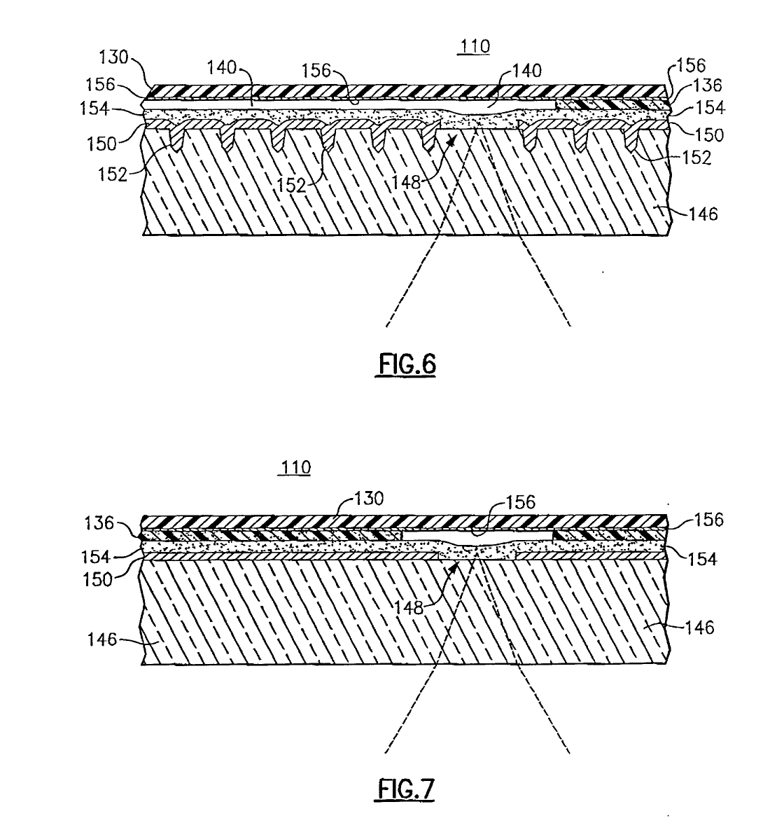

FIG. 6 is a partial longitudinal cross sectional view of the reflective optical bio-disc shown in FIGS. 3, 4, and 5 illustrating a wobble groove formed therein;

FIG. 7 is a partial cross sectional view taken perpendicular to a radius of the reflective optical bio-disc illustrated in FIGS. 3, 4, and 5 showing a flow channel formed therein;

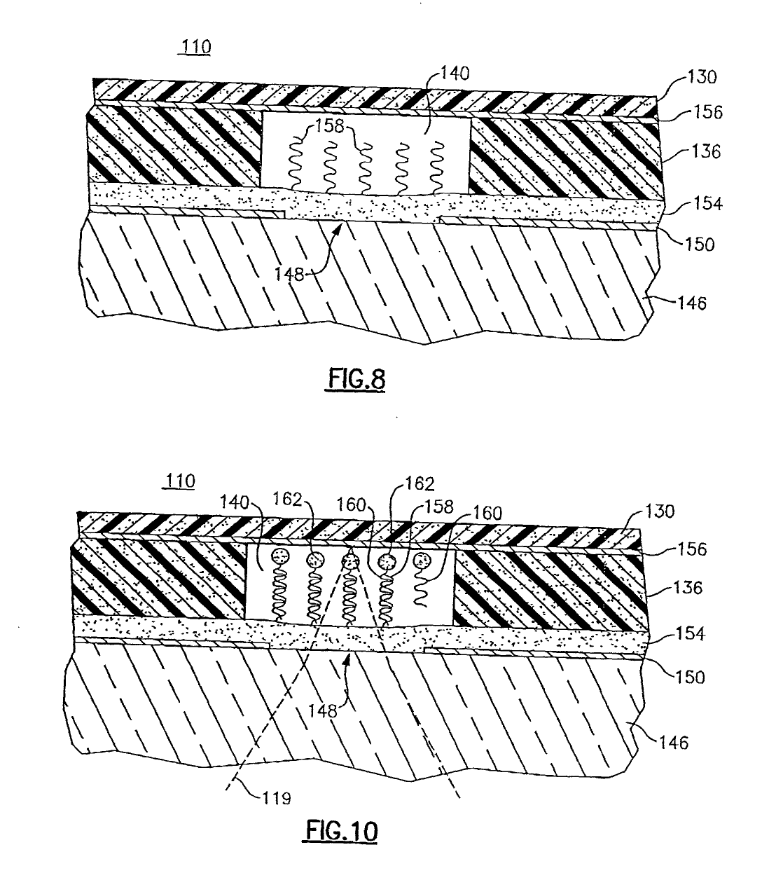

FIG. 8 is an enlarged partial cross sectional view similar to FIG. 7, further showing capture DNA in the flow channel;

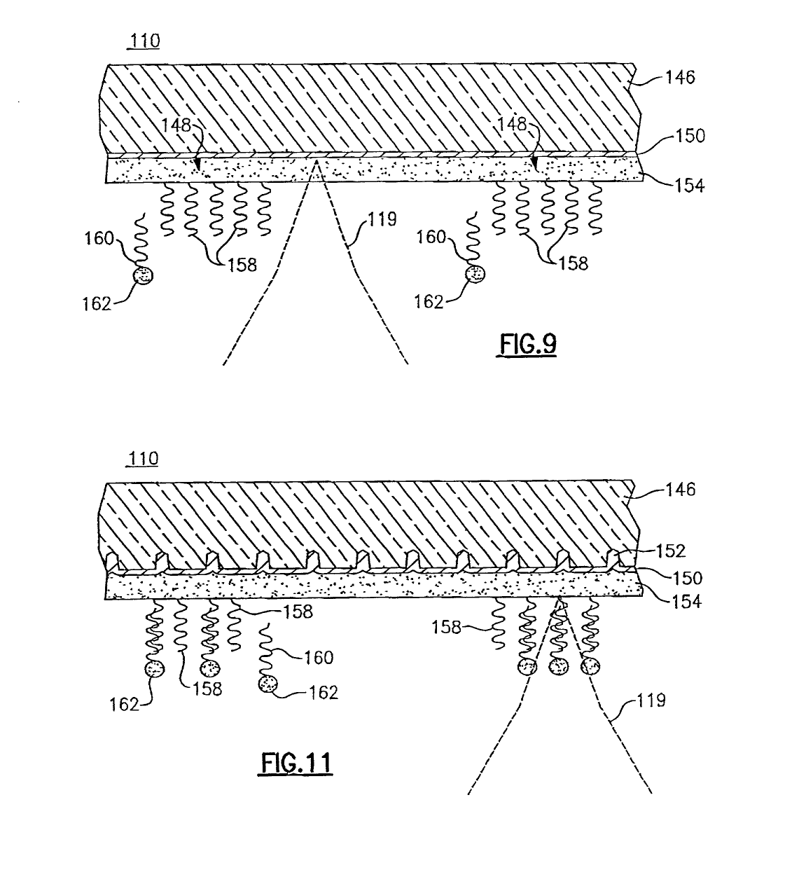

FIG. 9 is a partial cross sectional view of an alternative “open” embodiment of the present invention, showing capture DNA bound to active layer formed as a proximal layer relative to an interrogation beam;

FIG. 10 is an enlarged view similar to FIG. 8, further showing target DNA bound to a reporter bead hybridizing with the capture DNA;

FIG. 11 is an enlarged view similar to FIG. 9, further showing target DNA bound to a reporter bead hybridizing with the capture DNA;

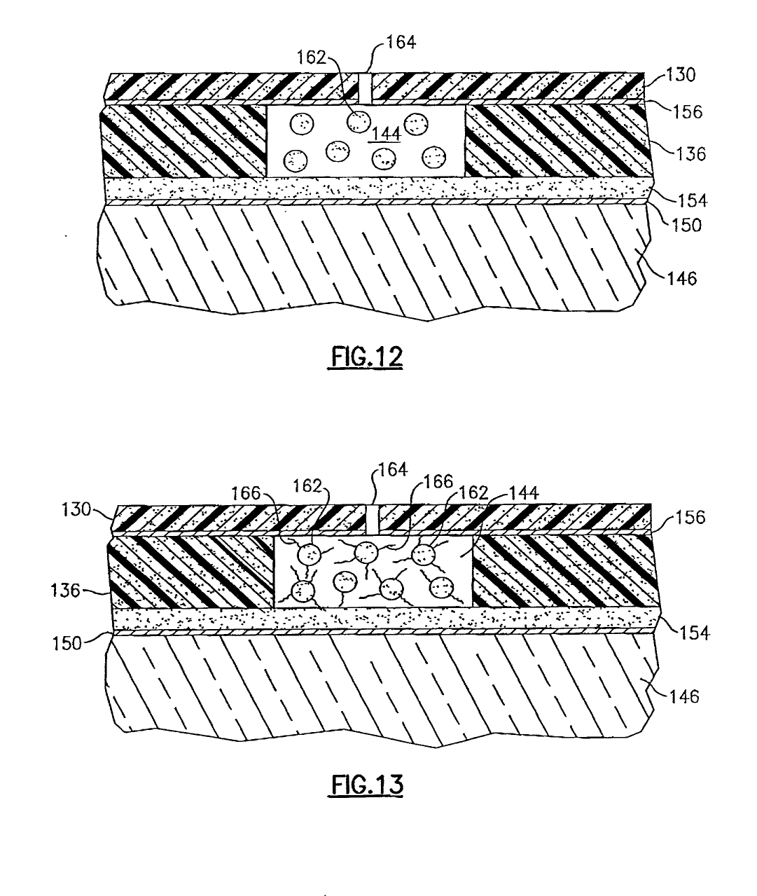

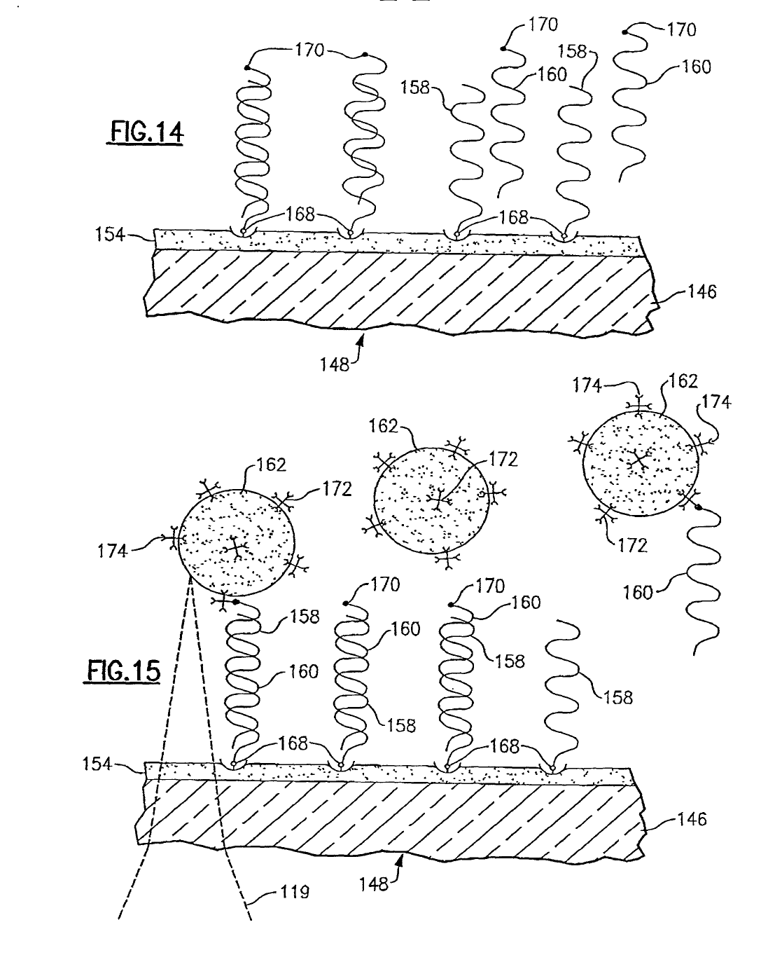

FIG. 12 is a cross sectional view of a mixing chamber, showing an input port and reporter beads pre-loaded into the mixing chamber;

FIG. 13 is a cross sectional view of a mixing chamber, similar to FIG. 12, showing an alternative embodiment of the present invention in which signal DNA is linked to reporter beads;

FIG. 14 is a detailed partial cross sectional view showing biotinylated target DNA hybridizing to complementary capture DNA bound to an active layer of the optical disc;

FIG. 15 is a detailed partial cross sectional view similar to FIG. 14, further showing streptavidin or NeutrAvidin coated reporter beads binding to biotin on target DNA;

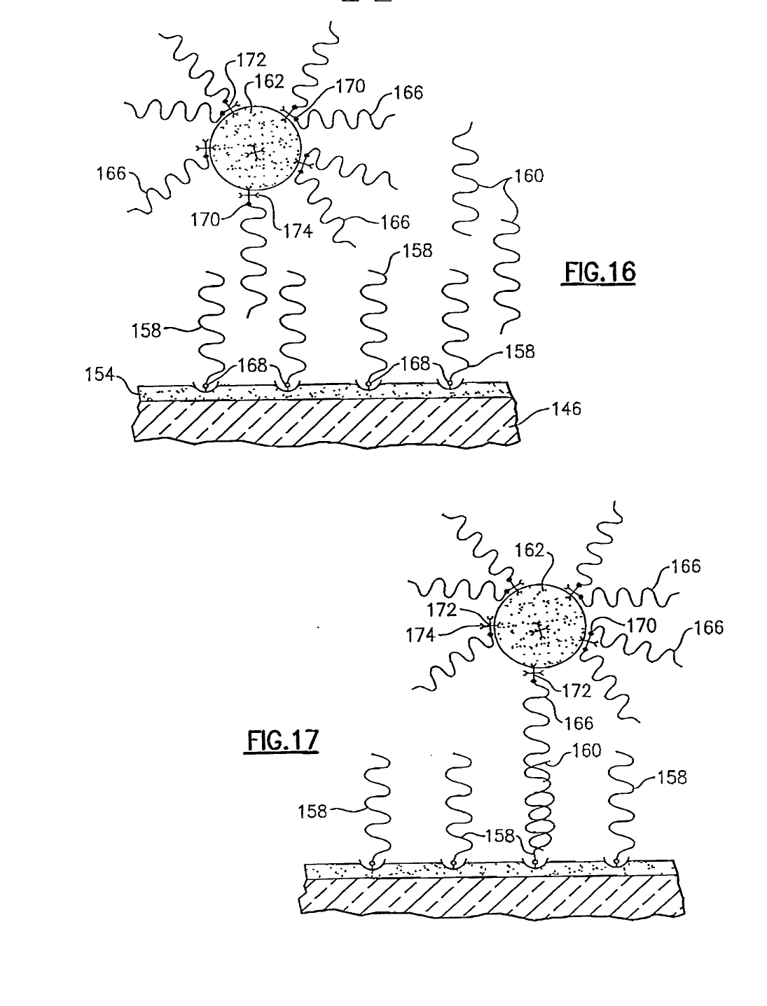

FIG. 16 is a detailed partial cross sectional view showing capture DNA bound to the active layer, a streptavidin or NeutrAvidin coated reporter bead complexed with biotinylated signal DNA, and target DNA, which is partially complementary to the capture DNA and partially complementary to the signal DNA;

FIG. 17 is a detailed partial cross sectional view similar to FIG. 16, further showing the target DNA hybridizing to both capture DNA and signal DNA, to thereby complex the reporter bead with the capture DNA;

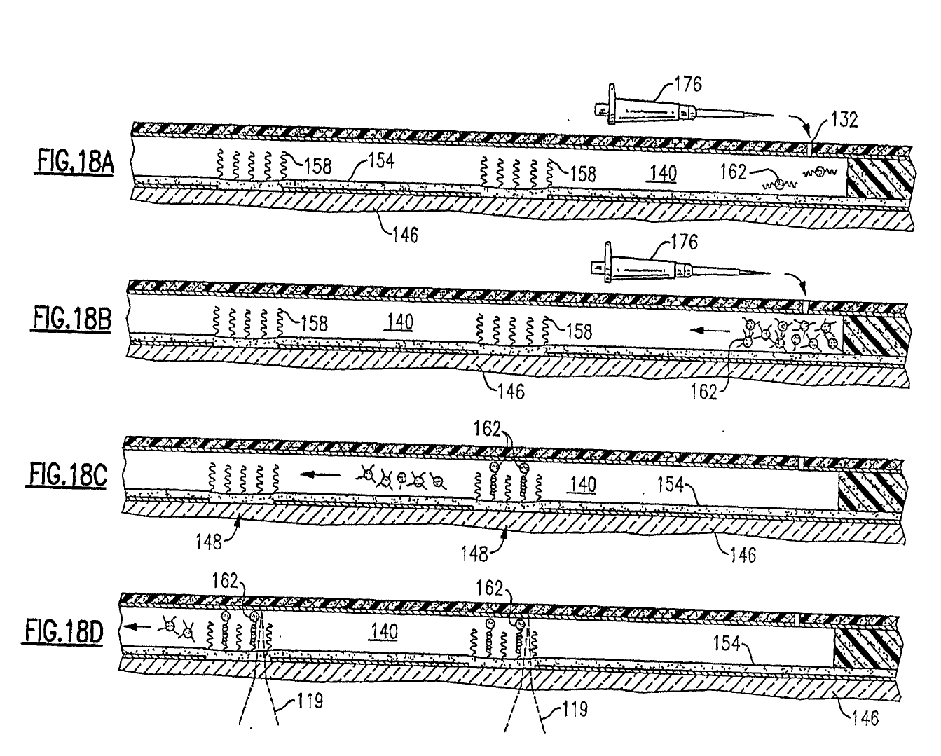

FIGS. 18A-D show a longitudinal cross-section of a flow channel, illustrating a method according to the present invention for detecting or determining the presence of target DNA in which the target DNA is linked to reporter beads prior to introduction into the flow channel;

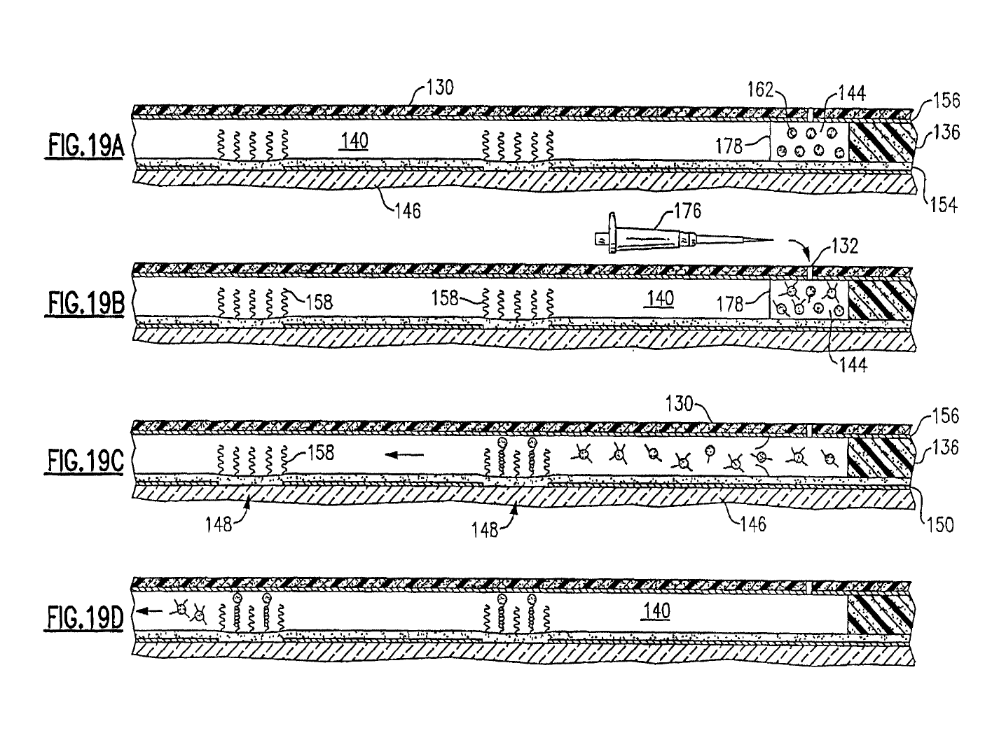

FIGS. 19A-D show a longitudinal cross-section of a flow channel, illustrating another embodiment of a method according to the present invention for detecting or determining the presence of target DNA in which reporter beads and target DNA are added to a mixing chamber containing a breakaway wall, and allowed to interact before entering the flow channel;

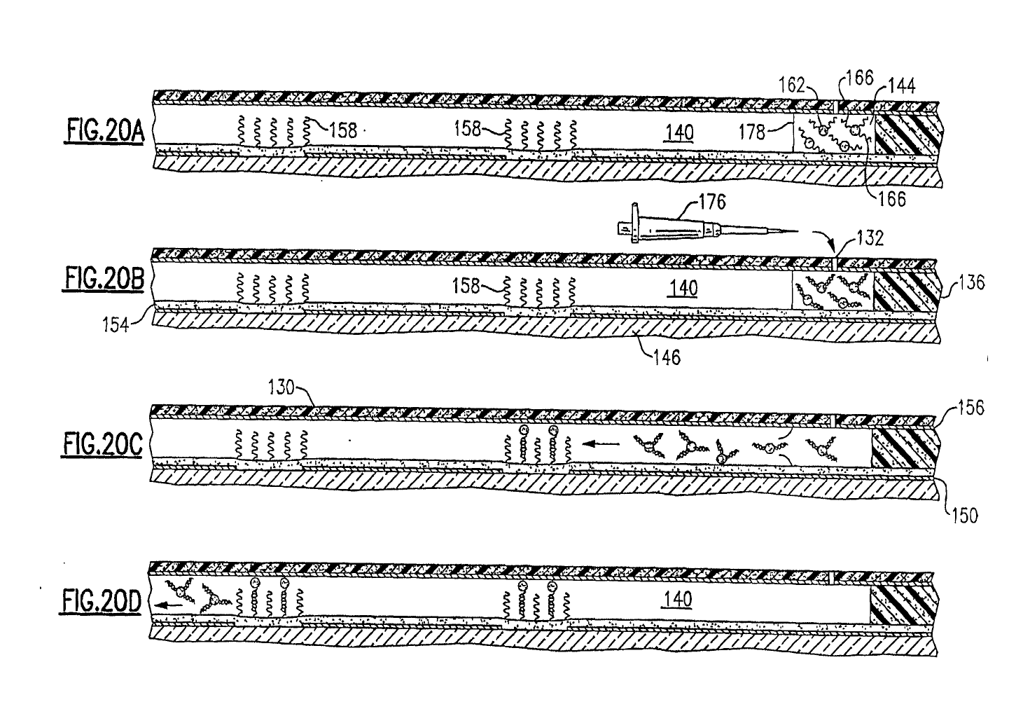

FIGS. 20A-D show a longitudinal cross-section of a flow channel, illustrating another embodiment of a method according to the present invention for detecting or determining the presence of target DNA similar to that shown in FIG. 19, except reporter beads are bound to signal DNA, which hybridizes with target DNA in the mixing chamber prior to entering the flow channel;

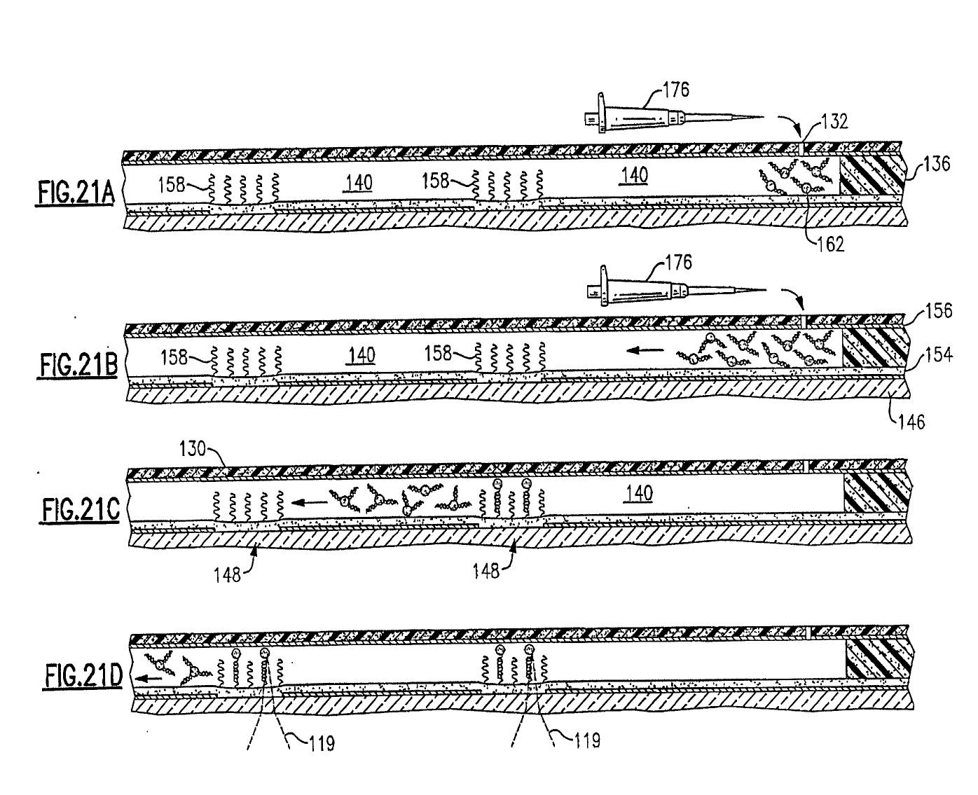

FIGS. 21A-D show a longitudinal cross-section of a flow channel, illustrating another embodiment of a method according to the present invention for detecting or determining the presence of target DNA similar to that shown in FIG. 18, except target DNA is hybridized with signal DNA bound to reporter beads prior to introduction into the optical disc flow channel;

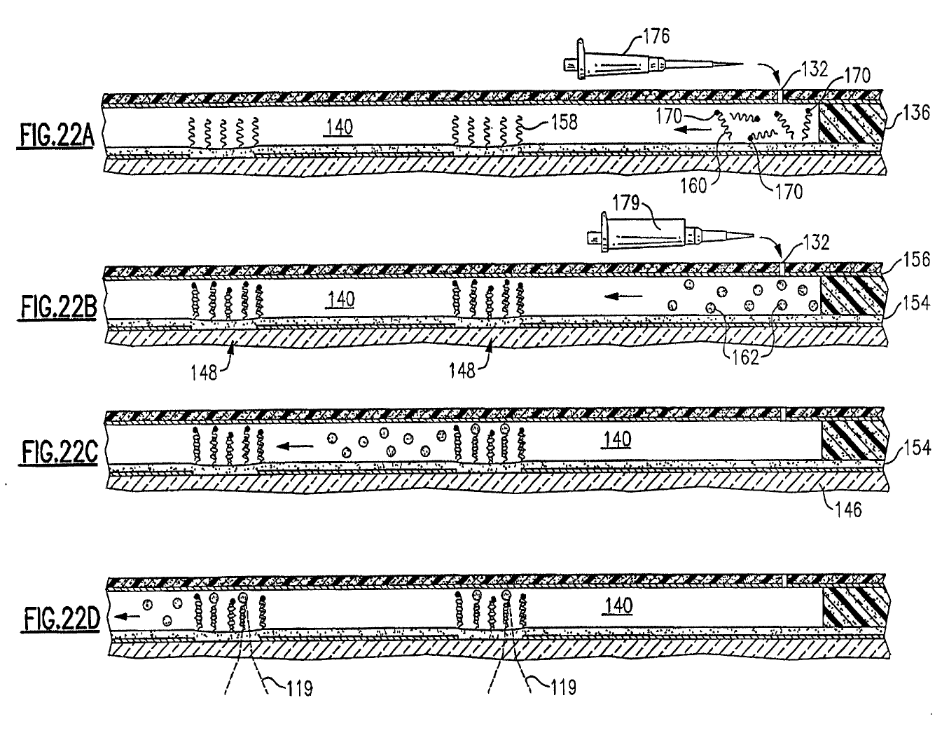

FIGS. 22A-D show a longitudinal cross-section of a flow channel, illustrating another embodiment of a method according to the present invention for detecting or determining the presence of target DNA, in which biotinylated target DNA is hybridized to capture DNA prior to the introduction of streptavidin or NeutrAvidin coated reporter beads into the flow channel;

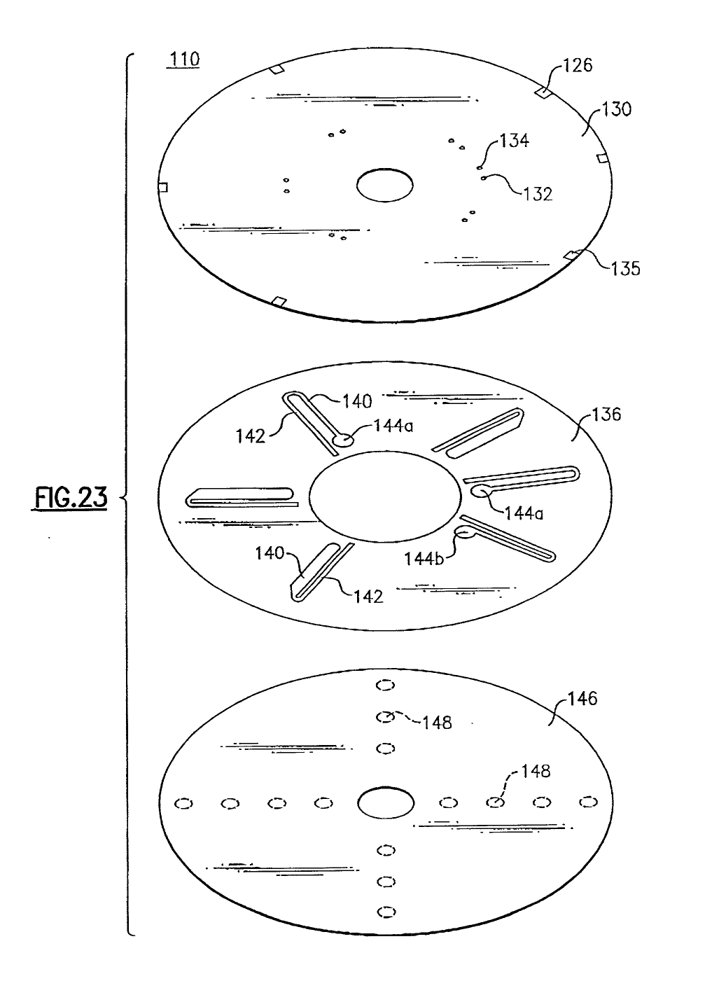

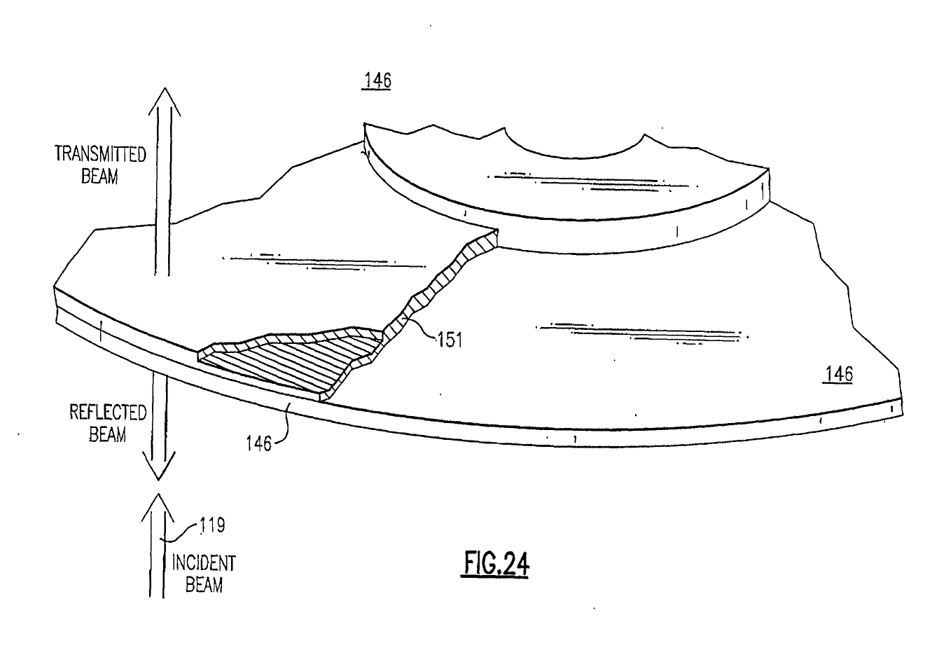

FIG. 23 is an exploded perspective view of a transmissive bio-disc as employed in conjunction with the present invention;

FIG. 24 is a perspective view representing the disc shown in FIG. 23 with a cut-away section illustrating the functional aspects of a semi-reflective layer of the disc;

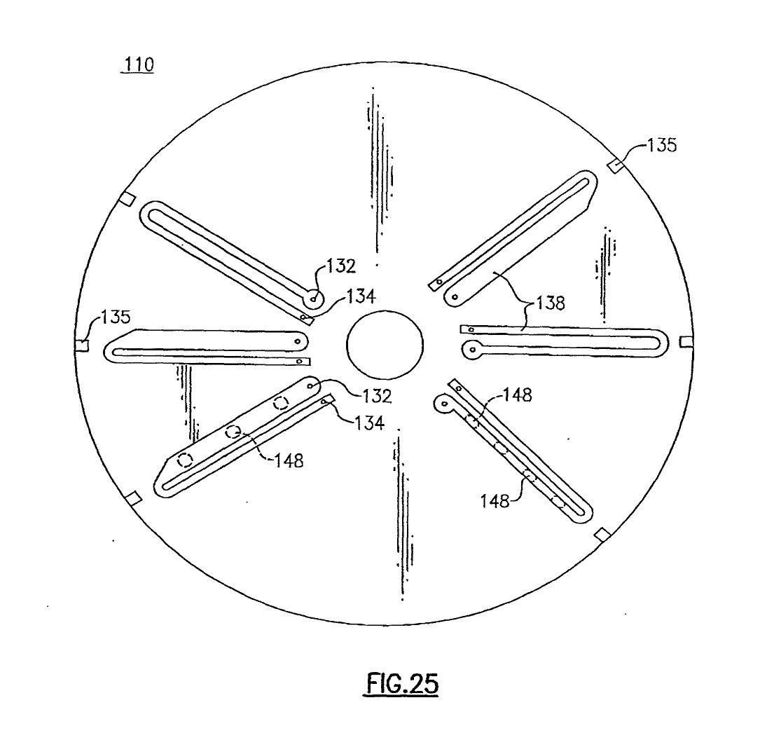

FIG. 25 is a top plan view of the disc shown in FIG. 23;

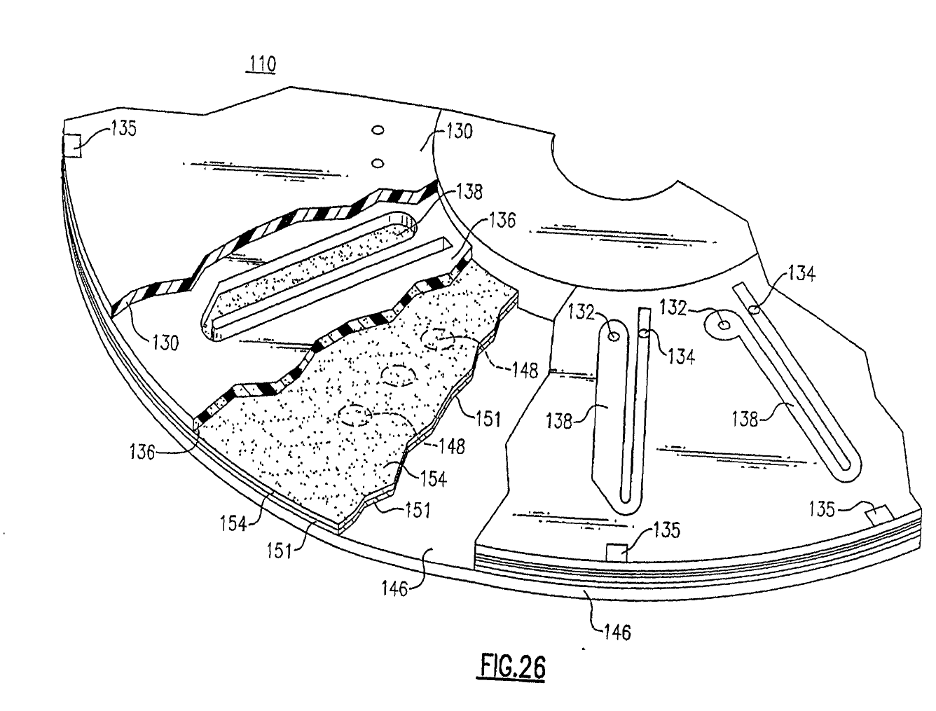

FIG. 26 is a perspective view of the disc illustrated in FIG. 23 with cut-away sections showing the different layers of the disc including the type of semi-reflective layer shown in FIG. 24;

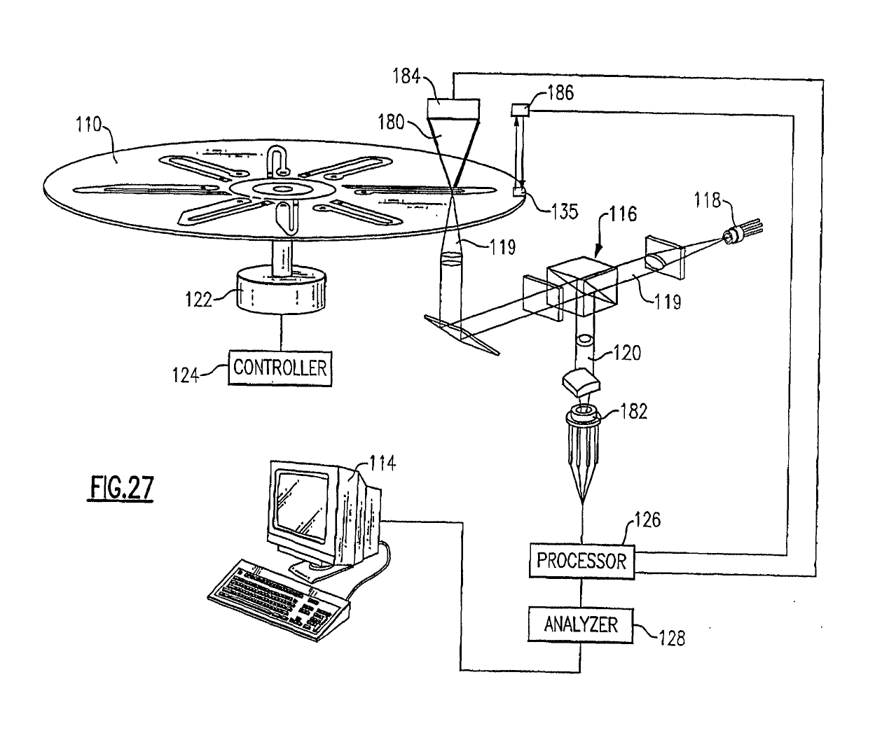

FIG. 27 is a perspective and block diagram representation illustrating the system of FIG. 1, for use with transmissive optical bio-discs;

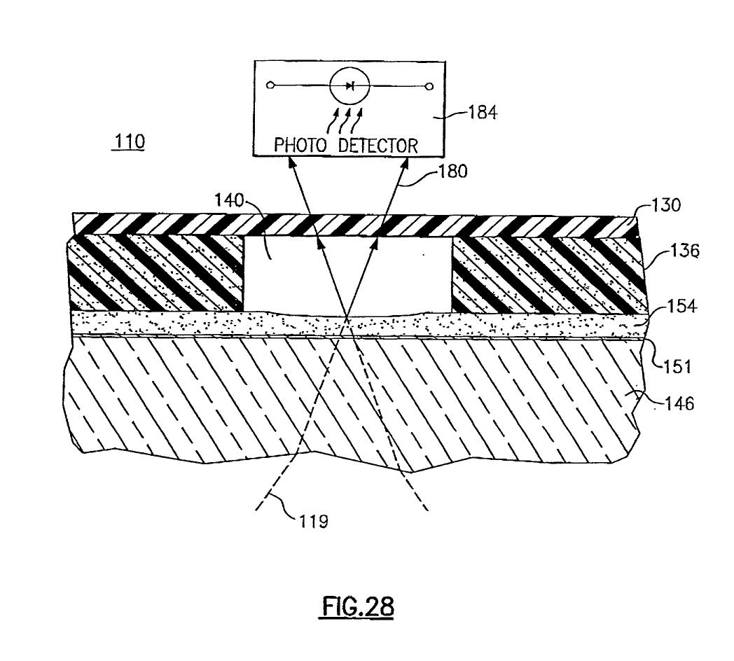

FIG. 28 is a partial cross sectional view taken perpendicular to a radius of the transmissive optical bio-disc illustrated in FIGS. 23, 24, 25 and 26 showing a flow channel formed therein and a top detector;

FIG. 29 is a partial longitudinal cross sectional view of the transmissive optical bio-disc illustrated in FIGS. 23, 24, 25 and 26 showing a wobble groove formed therein and a top detector;

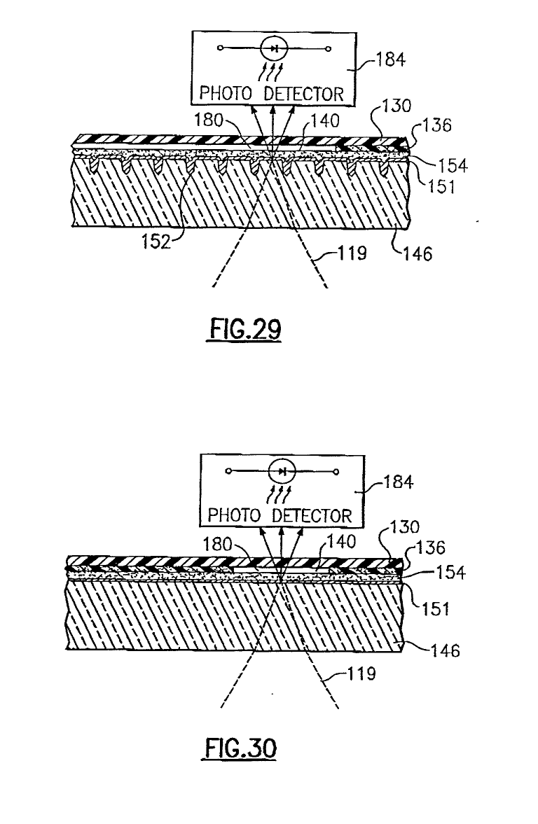

FIG. 30 is a view similar to FIG. 28 showing the entire thickness of the transmissive disc and the initial refractive property thereof;

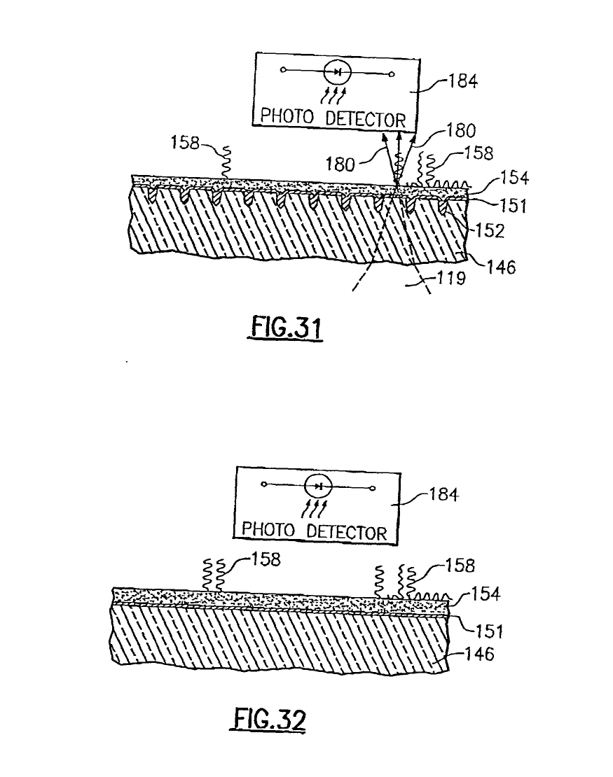

FIG. 31 is a partial cross sectional view of an alternative “open” embodiment of a transmissive disc of the present invention, showing capture DNA bound to active layer formed as a distal layer relative to an interrogation beam and further showing tracking grooves formed in the substrate;

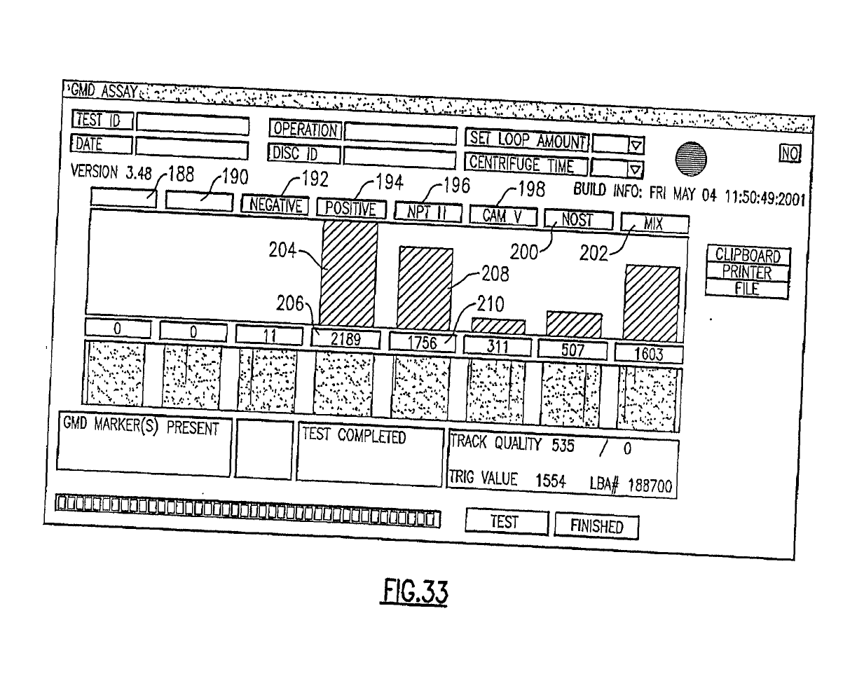

FIG. 32 is a cross-sectional view of the disc shown in FIG. 31, taken longitudinally along one of the tracking grooves;

FIG. 33 is a sample display readout showing the results of a DNA assay of the present invention.

### DETAILED DESCRIPTION OF THE INVENTION

Brief Overview of the Assay. In the method of the present invention, reporter bead binding is used to detect the presence of an analyte nucleic acid (DNA or RNA) in a microchannel on a bio-disc. The analyte is immobilized in a target zone in the microchannel through hybridization, either direct or indirect, to capture DNA bound to the bio-disc active layer. Reporter beads bind to the analyte, either before or after hybridization with the capture DNA as, for example, through the interaction of streptavidin on the reporter bead and biotin on the analyte. Subsequent to hybridization, “free” reporter beads, those not complexed with analyte and thus with capture DNA, are washed away, as, for example, by a stream of buffer or by centrifugation, leaving only reporter beads complexed with analyte in the target zone.

Bead binding generates a localized and specific signal, which may be detected and quantified by the optical bio-disc reader utilized in conjunction with the inventions hereof. In one embodiment, bead binding is detected by changes in reflectivity of an interrogation beam. In another embodiment, bead binding is detected by changes in the transmittance of the interrogation beam. Alternatively, beads may be labeled with fluorescent markers, in which case bead binding is detected via fluorescent signals using a fluorometer. This embodiment may use a fluorescence-type optical disc reader, as, for example, where the drive shown in FIG. 1 is implemented with hardware and optics for fluorescence detection.

In still another embodiment, target or signal DNA may be labeled with fluorescent markers. In this embodiment, reporter beads are not required as fluorescence is detected directly from the target or signal DNA.

Analytes. The present invention is directed to the detection and analysis of target nucleic acids present in test samples. Target nucleic acids suitable for use with the present invention include both deoxyribonucleic acid (DNA) and ribonucleic acid (RNA), including mRNA, rRNA and tRNA. Target nucleic acid may be used directly from a biological sample, but preferably is amplified prior to testing via polymerase chain reaction (PCR) or isothermal amplification to generate amplicons. If using PCR for amplification, RNA may first be reverse transcribed into DNA using techniques well known in the art. Target nucleic acid may be single stranded or double stranded. If double stranded, the nucleic acid may be denatured prior to hybridization with capture DNA.

In one embodiment of the present invention, primers labeled with biotin are used in PCR reactions to yield biotin-labeled target DNA amplicons, which are then tested in the bio-disc assay as described below. Amplicons of various lengths are suitable for use in the present invention, with a preferred length from about 20 bases (or base pairs) to about 4000 bases (or base pairs), more preferably from about 200 to about 400 bases or base pairs.

The present invention may be used to detect nucleic acids in a wide variety of biological samples, including but not limited to bodily fluids such as whole blood, serum, plasma, saliva, urine, lymph, spinal fluid, tears, mucous, semen and the like, agricultural products, food items, waste products, environmental samples, such as soil and water samples, or any other sample containing, or suspected of containing, nucleic acids. For example, the present invention may be used to detect the presence of particular strains of microorganisms, such as viruses or bacteria, in body fluids or environmental samples, by detecting the presence of particular nucleic acid sequences in the sample. In another example, the present invention may be used to detect the presence of genetically modified agricultural products in food items. Other uses of the present invention will be apparent to those of skill in the art.

Capture DNA. Capture DNA oligonucleotides, or probes, are immobilized onto a bio-disc and are hybridized to target DNA or RNA to thereby “capture” the target nucleic acid, with associated reporter beads, in the target zone for detection. Capture DNA may be single strand or partially double strand near the attachment point to the active layer on the bio-disc. One preferred embodiment of the capture DNA includes double strand DNA at the active layer because the double strand has been found to more effectively project the capture probe erectly or upwardly from the active layer as compared to ssDNA in some instances. In the case of a partially double-stranded capture DNA, an extension of ssDNA is employed so that hybridization may occur with a target DNA. The sequence of the capture DNA is selected so as to hybridize directly with target DNA or RNA, thereby forming a complex comprising capture DNA, target DNA or RNA and reporter beads bound thereto.

In an alternative embodiment described below, signal DNA may also be present in this complex. In this embodiment, a portion of the signal DNA sequence is complementary to the target DNA or RNA, but is not complementary to the capture DNA. As one portion of the target DNA can hybridize with capture DNA, while a different portion can hybridize with signal DNA, a complex may form in which the target DNA acts as a “bridge” between the capture DNA and signal DNA. Reporter beads may be bound to the signal DNA either before or after formation of this complex.

In a preferred embodiment of the present invention, the capture oligonucleotides are connected to an NH₂ group, and the disc surface is coated with a layer of modified polystyrene, preferably polystyrene-co-maleic anhydride. The NH₂ group covalently binds to the maleic anhydride, thereby attaching the DNA capture oligonucleotide to the disc surface in the target zone. Such aminated capture DNA is commercially available from, for example, Operon (Alameda, Calif.), Annovis (Aston, Penn.), or BioSource International (Camarillo, Calif.).

Signal DNA. In one embodiment of the present invention, target DNA hybridizes directly with capture DNA bound to the active layer of a bio-disc. In an alternative embodiment, target DNA may be used as a “bridge” between signal DNA and the capture DNA. In this embodiment, the sequence of the signal DNA is selected so as to contain a region which is complementary to the target DNA, but which contains no sequence complementary to the capture DNA, such that the signal DNA will not form a complex with the capture DNA in the absence of target DNA. The target DNA contains a first region of complementary sequence to the capture DNA, permitting hybridization of the first region of the target DNA to the capture DNA, and a second region of complementary sequence to the signal DNA, permitting hybridization of the signal DNA to the second region of the target DNA, thereby linking or bridging the signal DNA to the capture DNA.

The target DNA may be of any length suitable to effectively immobilize the signal DNA to the capture DNA. Typically, target DNA amplicons used with signal DNA are from about 10 bases to about 100 bases in length, preferably from about 20 bases to about 60 bases in length. Typically, the target DNA amplicons have an overlap of from about 20 bases to about 40 bases with the signal DNA and an overlap of from about 20 bases to about 40 bases with the capture DNA. Preferably, the target DNA amplicon has a GC (guanine and cytosine) content greater than 50%, although one skilled in the art will appreciate that GC content and length of the target DNA amplicon may be modulated to effectuate stable hybridization to the signal and capture DNA.

In one embodiment of the present invention, signal DNA is labeled with an affinity agent, such as biotin or an amino group, to permit binding to reporter beads, as, for example, through covalent interaction (as with an amino group) or via biotin/streptavidin interactions (with streptavidin coated beads). Alternatively, signal oligonucleotides may be thiolated, for direct binding to colloidal gold beads. Suitable signal oligonucleotides, including oligonucleotides with amino groups, biotin or thiol groups, may be commercially acquired from, for example, Operon (Alameda, Calif.), Annovis (Aston, Penn.), or BioSource International (Camarillo, Calif.).

Reporter Beads. The beads of the instant method are preferably spherical in shape and are typically from about 500 nm to about 5 μm in diameter, preferably from about 1 μm to about 3 μm in diameter. As will be appreciated by one skilled in the art, the size of the bead used in the assay may affect the rate of revolution of the bio-disc used during mixing and wash steps, as described below. Generally, the greater the mass of the bead, the slower the rate of revolution employed.

The beads are preferably formed from polystyrene, but may be formed from a number of suitable materials, such as colloids, including colloidal gold, glass, polymethylmethacrylate and magnetic beads. If colloidal beads are used, an additional enhancement step is used, in which the DNA/reporter bead complex is treated with an ionizing wash of ions, such as gold or silver ions to enhance the colloidal bead.

In one embodiment, the beads are coated with streptavidin, for use with biotinylated target or signal DNA. Alternatively, carboxylated beads may be covalently linked to aminated signal or target DNA.

In a preferred embodiment, reporter beads linked to target DNA/capture DNA complexes are detected by changes in reflectivity, or transmittance, of an incident interrogatory beam. In an alternative embodiment, reporter beads may be labeled with fluorescent markers, in which case reporter beads are detected by measurement of fluorescence signal on the disc surface using a FluorImager (Molecular Dynamics). Suitable fluorescent markers include, for example, the cyanine dyes.

Beads suitable as a starting material in accordance with practice of the present invention are commercially available from suppliers such as Molecular Probes, Inc. (Eugene, Oreg.), Polysciences, Inc. (Warrington, Penn.) or Spherotech, Inc. (Libertyville, Ill.).

Further objects of the present invention together with additional features contributing thereto and advantages accruing therefrom will be apparent from the following description of preferred embodiments of the present invention which are shown in the accompanying drawing Figures with like reference numerals indicating like components throughout.

With reference to FIG. 1, there is shown a perspective view of an optical bio-disc 110 according to the present invention. The present bio-disc 110 is shown in conjunction with an optical disc drive 112 and display monitor 114.

FIG. 2 is a partial pictorial representation in perspective and block diagram illustrating optical components 116, laser source 118 which produces an incident or interrogation beam 119, and a return beam 120. The return beam 120 is returned or reflected from the bio-disc 110 or investigational features such as reporters that reside on or in the disc 110. FIG. 2 also illustrates a drive motor 122 and a controller 124 for controlling the rotation of the bio-disc 110 optical. FIG. 2 further illustrates a processor 126 and analyzer 128 for processing the return beam 120. The detection, signal processing, and imaging techniques are further described in commonly assigned U.S. Provisional Application Nos. 60/270,095, filed Feb. 20, 2001; and 60/292,180, filed May 18, 2001, both entitled Signal Processing Apparatus and Methods for Obtaining Signal Signatures of Investigational Features Detected on a Surface of an Optical Disc Assembly; and commonly assigned U.S. Application Ser. No. 10/008,156, filed Nov. 9, 2001, entitled Disc Drive System and Methods for Use with Bio-discs.

An exploded perspective view of the principle structural elements of the present bio-disc 110 is shown in FIG. 3. A cap portion 130 includes an inlet port 132 and a vent port 134. The cap portion 130 may be formed from polycarbonate and is preferably coated with a reflective layer on the bottom thereof as viewed form the perspective of the Figure. The second element shown in FIG. 3 is a plastic film and adhesive member or membrane 136 having fluidic circuits or U-channels 138 formed therein. The fluidic circuits 138 are formed by stamping or cutting the membrane to remove plastic film and form the shapes as indicated. Each of the fluidic circuits 138 includes a flow channel 140 and a return channel 142. Some of the fluidic circuits 138 illustrated in FIG. 3 include a mixing chamber 144. Two different types of mixing chambers are illustrated. The first is a symmetric mixing chamber 144a which is symmetrically formed relative to the flow channel 140. The second is an off-set mixing chamber 144b. The off-set mixing chamber 144b is formed to one side of the flow channel 140 as indicated. The third element illustrated in FIG. 3 is a substrate 146 including target or capture zones 148. The substrate 146 is preferably made of polycarbonate and has a reflective layer deposited on the top thereof. The target zones 148 are formed by removing the reflective layer in the indicated shape or alternatively in any desired shape. Alternatively, the target zones 148 may be formed by a masking technique that includes masking the target zone area before applying the reflective layer. The reflective layer may be formed from a metal such as aluminum or gold.

With reference to FIG. 4, there is shown a top plan view of the optical bio-disc 110 illustrated in FIG. 3 with the reflective layer on the cap portion 130 shown as transparent to reveal the fluidic circuits 138 and target zones 148 as situated within the disc.

FIG. 5 is an enlarged perspective view of the optical bio-disc 110 according to one embodiment of the present invention having a portion of the various layers thereof cut away to illustrate a partial sectional view of each principle, layer, substrate, coating, or membrane. FIG. 5 shows the substrate 146 which is coated with a reflective layer 150. An active layer 154 is applied over the reflective layer 150. The active layer 154 may be formed from a modified polystyrene or such as, for example, polystyrene-co-maleic anhydride. As illustrated in this embodiment, the plastic adhesive member 136 is applied over the active layer 154. The exposed section of the plastic adhesive member 136 illustrates the cut out or stamped U-shaped form that creates the fluidic circuits 138. The final principle structural layer in this embodiment of the present bio-disc 110 is the cap portion 130. As illustrated in FIG. 5, the cap 130 includes a reflective surface 156 on the bottom thereof. The reflective surface 156 may be made from a metal such as aluminum or gold. In this manner, the interrogation beam 119 (FIG. 2) from the laser source 118 (FIG. 2) may be directed through the target zones 148 and into the fluidic circuit 138, and thereafter reflected from the reflective surface 156 on the cap 130 to form or contribute to the return beam 120 (FIG.2). The reflective surface 156 may be applied to the entire bottom surface of the cap 130 or alternatively only above the target zones 148 as needed to reflect or return the interrogation beam 119.

A perspective view of a cross section of one embodiment of the bio-disc 110 according to the present invention is shown in FIG. 6. FIG. 6 includes the substrate 146 and the reflective layer 150. The reflective layer 150 may be made from a metal such as aluminum or gold. The substrate 146 in this embodiment includes a series of grooves 152. The grooves 152 are in the form of a spiral extending from near the center of the disc toward the outer edge. The grooves 152 are implemented so that the interrogation beam 119 (FIG. 2) may track long the spiral. The spiral groove in a recordable disc contains a dye rather than pits and lands which are typically employed in a prerecorded CD, for example. A typical recordable disc includes a spiral groove having a characteristic shape along the length thereof. This type of groove is known as a “wobble groove”. The wobble groove is formed by a bottom portion having undulating or wavy side walls. A raised or elevated portion separates adjacent grooves in the spiral. The reflective layer 150 applied over the grooves 152 in this embodiment is, as illustrated, conformal in nature. FIG. 6 also shows the active layer 154 applied over the reflective layer 150. As shown in FIG. 6, the target zone 148 is formed by removing an area or portion of the reflective layer 150 at a desired location or, alternatively, by masking the desired area prior to applying the reflective layer 150. As further illustrated in FIG. 6, the plastic adhesive member 136 is applied over the active layer 154. FIG. 6 also shows the cap portion 130 and the reflective surface 156 associated therewith. Thus when the cap 130 is applied to the plastic adhesive member 136 including the desired cut-out shapes, the flow channel 140 is thereby formed.

FIG. 7 is a cross sectional view of another embodiment of the bio-disc 110 according to the present invention. This embodiment does not include the recordable-CD or DVD wobble groove. In this embodiment, the substrate 146 is smooth. The reflective layer 150 is formed thereon and the target zone 148 is formed in a similar manner. FIG. 7 also shows the active layer 154, the plastic adhesive member 136, and the reflective surface 156 of the cap 130.

A view similar to FIG. 7 enlarged to illustrate capture DNA 158 attached to the active layer 154 within the target zone 148 is shown in FIG. 8. The capture DNA 158 in this embodiment is attached to the active layer 154 by applying a small volume of capture DNA solution to the active layer 154 to form clusters of capture DNA within the area of the target zone 148.

With reference to FIG. 9, there is shown an alternate embodiment of the present optical bio-disc which utilizes an open-face or open-disc format. In this embodiment, the substrate 146 is implemented as a distal layer relative to the interrogation beam 119. The reflective layer 150 is next provided as illustrated. The bottom layer or proximal layer relative to the beam 119 in this embodiment is provided by the active layer 154. In this embodiment, the capture DNA 158 may be depended downwardly when the disc 110 is loaded in the drive 112 (FIG. 1). In this open-face format, the target DNA 160 and reporters 162 are brought into proximity with the capture DNA 158 by a variety of different methods which include, for example, depositing a test sample on the disc with a pipette. The target zones 148 in this embodiment are simply formed by applying a small volume of capture DNA solution to the active layer 154 to form clusters of capture DNA in desired locations on the active layer 154 as illustrated.

FIG. 10 is a view similar to FIG. 8 illustrating the flow channel 140 and target zone 148 after hybridization of target DNA 160 with the capture DNA 158. FIG. 10 also shows reporters 162 as employed in the present invention. One embodiment of the reporters 162 includes beads or plastic micro-spheres. These beads may be made of polystyrene or gold and are coated with specified substances that have an affinity for the target DNA. FIG. 10 also shows use of the interrogation beam 119 as directed into the target zone 148 to search for reporters 162.

An alternate open-face format according to the present invention is shown in FIG. 11. In this embodiment, the tracking grooves 152 are employed. The use and operation of the bio-disc 110 according to this embodiment of the present invention, is similar to that described in conjunction with FIGS. 6 and 9.

FIG. 12 is a cross sectional view illustrating a pre-loaded mixing chamber 144. Access to the mixing chamber 144 is achieved by an inlet port 164. The mixing chamber 144 illustrated in FIG. 12 is pre-loaded with reporters 162.

A view similar to FIG. 12 showing the mixing chamber 144 pre-loaded in an alternate manner with reporters 162 that include signal DNA 166 is shown in FIG. 13. The signal DNA is illustrated as ssDNA but may also include some dsDNA. The signal DNA 166 is non-complementary to the capture DNA 158 (shown in FIG. 8), while the target DNA 160 in this embodiment is complementary to both the signal DNA 166 and the capture DNA 158.

With reference to FIG. 14, a detailed partial cross sectional view is shown with the active layer 154 and the substrate 146 of the present bio-disc 110. FIG. 14 also illustrates that the capture DNA 158 is attached to the active layer 154 by use of an amino group 168 which is made an integral part of the capture DNA 158. As indicated, the capture DNA is situated within the target zone 148. The bond between the amino group 168 and the capture DNA 158, and the amino group 168 and the active layer 154 is sufficient so that the capture DNA 158 remains attached to the active layer 154 within the target zone 148, when the disc is rotated. The amino group 168 may preferably include NH2. A thiol group may alternatively be employed in place of the amino group 168. FIG. 14 also depicts the target DNA 160. In this embodiment of the present invention, the target DNA includes the addition of an affinity agent 170 such as biotin. As the target DNA 160 flows toward the capture DNA 158 and is in sufficient proximity thereto, hybridization occurs between the target DNA 160 and the capture DNA 158.

A view similar to FIG. 14 after introduction of the reporters 162 is shown in FIG. 15. As illustrated, the reporters are coated with a binding agent 172 that includes receptors 174. The binding agent 172 may include streptavidin or NeutrAvidin. As illustrated in FIG. 15, the target DNA 160 hybridizes with capture DNA 158 and the affinity agent 170 links with the receptor 174 of the binding agent 172 to maintain the reporter 162 within the target zone 148. In this manner, the interrogation beam 119 may be introduced into the flow channel 140 (FIGS. 3, 4, and 5) through the target zone 148 to detect the presence of the reporter 162 to thereby determine the presence of the target DNA in a test sample of DNA. According to one aspect of the present invention, linking may first occur between the affinity agent 170 and the receptor 174, followed by hybridization between the capture DNA 158 and the target DNA 160. According to another aspect of this embodiment, hybridization between the capture DNA 158 and the target DNA 160 including the affinity agent 170 may be allowed to occur first, followed by linking between the affinity agent 170 and the receptor 174. In either case, the reporters are maintained within the target zone 148 as desired.

FIG. 16 is a detailed partial cross sectional view showing the active layer 154 and the substrate 146 of the present bio-disc 110 according to the embodiment utilizing the signal DNA 166 attached to the reporters 162. In this embodiment, the signal DNA is linked to the reporter 162 by use of the binding agent 172 and the receptors 174 associated therewith. In this embodiment, the affinity agent 170 is installed in the signal DNA 166 rather than the target DNA 160. As indicated above in reference to FIG. 13, the signal DNA 166 is non-complementary to the capture DNA 158, while the target DNA 160 in this embodiment is complementary to both the signal DNA 166 and the capture DNA 158.

A view similar to FIG. 16, after hybridization has occurred between the signal DNA 166 and the target DNA 160, as well as between the target DNA 160 and the capture DNA 158, is shown in FIG. 17.

FIGS. 18A-18D illustrate a method according to the present invention for detecting or determining the presence of target DNA in a sample of DNA in conjunction with the optical bio-disc according to the present invention. In FIG. 18A, a pipette 176 is loaded with a test sample of DNA that has been linked to reporters 162. The test sample is injected or deposited into the flow channel 140 through inlet or injection port 132. As the flow channel 140 is further filled with test sample, the reporters 162 begin to flow or move down the flow channel 140 as illustrated in FIG. 18B. When target DNA of a specific sequence is present in the test sample, the target DNA hybridizes with the capture DNA 158 as shown in FIGS. 18C and 18D. In this manner, the reporters 162 are retained within the target zones 148. Hybridization may be further facilitated by rotating the disc 110 so that the reporters 162 slowly move or tumble down the flow channel 140. Slow movement allows ample time for additional hybridization. After hybridization, the disc may be rotated to clear the target zones 148 of any unattached reporters 162. The interrogation beam 119 may then be scanned through the target zone 148 to determine the presence of reporters 162 as illustrated in FIG. 18D. In the event no target DNA is present in the test sample, all the reporters 162 are spun down the flow channel 140 when the disc is rotated. In this case, when the interrogation beam 119 is directed into the target zones 148, a negative reading will result thereby indicating that no target DNA was present in the sample.

With reference to FIGS. 19A-19D, another test method according to the present invention is illustrated, which utilizes an alternate embodiment of the optical bio-disc 110 according to this invention. FIG. 19A is a longitudinal cross sectional view of the flow channel 140. FIG. 19A shows a mixing chamber 144 including the reporters 162 in a pre-loaded configuration as discussed in connection with FIG. 12 above. The mixing chamber is sized so as to assist with fluid mixing of the test sample and the reporters. In this embodiment of the mixing chamber 144, there is provided a break-away retaining wall 178 which holds the reporters 162 within the mixing chamber 144 during the mixing procedure. FIG. 19B illustrates the pipette 176 depositing a test sample of DNA into the mixing chamber 144 via the inlet port 132. The test sample is to be tested for the presence of a target DNA with a particular sequence. After the test sample is deposited into the mixing chamber 144, the disc is rotated at a first predetermined rate to allow mixing and linking between the DNA test sample and the reporters 162 in a manner described in connection with FIG. 15. The drive may be equipped with control software or, alternatively, software instructions may be encoded on the bio-disc itself, to provide instructions to the controller 124 to rotate the disc forward and backward, or clockwise and counterclockwise, at a predetermined RPM and/or frequency to assist with mixing, such as is described in further detail in commonly assigned U.S. application Ser. No. 09/997,741, filed Nov. 27, 2001, and entitled Dual Bead Assays Including Optical Bio-discs and Methods Related Thereto.

After the test DNA is linked to the reporters 162, the disc is rotated at a second predetermined rate with causes the break-away retaining wall 178 to open as illustrated in FIG. 19C. Thereafter, the reporters 162 and test DNA linked thereto travel down the flow channel 140. When target DNA is present in the test sample, hybridization occurs between the target DNA and the capture DNA 158 as illustrated in FIGS. 19C and 19D. After any hybridization has occurred, the disc is further rotated to clear the target zones 148 of any excess or unattached reporters as illustrated in FIG. 19D. These excess or unattached reporters may thereby be directed into the bottom of the flow channel 140. Detection with the interrogation beam 119 is carried out as described above.

FIGS. 20A-20D illustrate yet another method according to the present invention which utilizes the alternate embodiment of the optical bio-disc 110 described in conjunction with FIGS. 19A-19D. FIG. 20A is a longitudinal cross sectional view of the flow channel 140. FIG. 20A shows the mixing chamber 144 including the reporters 162 and signal DNA 166 in a pre-loaded configuration as discussed in connection with FIG. 13 above. In this embodiment of the mixing chamber 144, the break-away retaining wall 178 holds the reporters 162 including the signal DNA 166 within the mixing chamber 144 during the mixing procedure. FIG. 20B illustrates the pipette 176 depositing a test sample of DNA into the mixing chamber 144 via the inlet port 132. The test sample is to be tested for the presence of a target DNA with a particular sequence. After the test sample is deposited into the mixing chamber 144, the disc is rotated at a first predetermined rate to allow mixing and hybridization between any target DNA in the test sample and the signal DNA linked to the reporters 162. Hybridization occurs in a manner similar to that described above in connection with FIGS. 16 and 17. As described with reference to FIG. 19, the drive may rotate the disc backwards and forwards at a predetermined frequency to assist with mixing.

After the any target DNA is hybridized with the signal DNA, the disc is rotated at a second predetermined rate with causes the break-away retaining wall 178 to open as illustrated in FIG. 20C. Thereafter, the reporters 162 and any target DNA hybridized thereto travel down the flow channel 140. When target DNA is present in the test sample, hybridization now occurs again between the target DNA and the capture DNA 158 as illustrated in FIGS. 20C and 20D. After any hybridization has occurred, the disc is further rotated to clear the target zones 148 of any excess or unattached reporters. Detection with the interrogation beam 119 is again carried out as described above.

FIGS. 21A-21D illustrate still yet another alternate test method according to the present invention. This method is similarly directed to determining the presence of target DNA in a sample of DNA and is practiced in conjunction with the optical bio-disc according to the present invention. In FIG. 21A, the pipette 176 is loaded with a test sample of DNA that has been mixed with reporters 162 including signal DNA 166. In the event target DNA is present in the test sample, hybridization has been allowed to occurred between the target DNA and the signal DNA. The test sample is injected or deposited into the flow channel 140 through inlet or injection port 132. As the flow channel 140 is further filled with test sample, the reporters 162 including the signal DNA and any target DNA begin to flow down the flow channel 140 as illustrated in FIG. 21B. When target DNA of a specific sequence is present in the test sample, the target DNA hybridizes with the capture DNA as shown in FIGS. 21C and 21D. In this manner, the reporters 162 are retained within the target zones 148. After hybridization, the disc may be rotated to clear the flow channel 140 of any unattached or excess reporters 162 as represented in FIG. 21D. The interrogation beam 119 may then be scanned through the target zones 148 to determine the presence of reporters 162 as illustrated in FIG. 21D. In the event no target DNA is present in the test sample, all the reporters 162 are flushed out of the target zones 148 when the disc is rotated. In this case, when the interrogation beam 119 is directed into the target zones 148, a negative reading will result thereby indicating that no target DNA was present in the sample.

FIGS. 22A-22D illustrate yet another additional method according to the present invention. This method utilizes the alternate embodiment of the optical bio-disc 110 described in conjunction with FIGS. 18A-18D. FIG. 22A is a longitudinal cross sectional view of the flow channel 140. FIG. 22A shows the pipette 176 depositing sample DNA including the affinity agent 170. As the flow channel 140 is filled with sample DNA 160, any target DNA will hybridize with the capture DNA 158 situated within the target zones 148 as shown in FIG. 22B. This occurs in a manner similar to that described above in connection with FIG. 14. As further shown in FIG. 22B, a second pipette 179 deposits reporters 162 into the flow channel 140 through the inlet port 132. The reporters 162 include the binding agent 172 and receptors 174 discussed in connection with FIG. 15. As the reporters 162 move down the channel, they link to the affinity agents 170 of any target DNA that has hybridized to the capture DNA 158 as illustrated in FIGS. 22C and 22D. This occurs in a manner similar to that described above in connection with FIG. 15. After any hybridization has occurred between any target DNA 160 and the capture DNA 158 and linking between the reporters 162 and the affinity agents 170, the disc is rotated to clear the target zones 148 of any excess or unattached reporters as also illustrated in FIG. 22D. These may be directed into the end of the flow channel 140 so that they are not present in the target zones 148 and thus detected by the interrogation beam 119. Detection with the interrogation beam 119 is again carried out as described above to determine the presence of any target DNA.

The above Figures describe an embodiment of the present invention using a reflective bio-disc, in which reporter beads are detected by changes in reflectivity of an interrogatory beam. In an alternative embodiment, described below, a transmissive bio-disc may be used in which reporter beads are detected by changes in transmittance of the interrogatory beam.

FIG. 23 is an exploded perspective view of the principle structural elements of a transmissive optical bio-disc 110 that may be used in the present invention. The principle structural elements include the cap portion 130, the adhesive member 136, and the substrate 146.

The cap portion 130 includes the inlet port 132 and the vent port 134. Optional trigger markings 135 may be included on the surface of a thin semi-reflective layer 150, as best illustrated in FIGS. 24 and 26. Trigger markings 135 may include a clear window in all three layers of the bio-disc, an opaque area, or a reflective or semi-reflective area encoded with information that sends data to the processor 126, shown in FIG. 27, which in turn interacts with the operative functions of the interrogation beam 199, shown in FIGS. 24 and 27.

The adhesive member 136 has fluidic circuits or U-channels formed therein. The fluidic circuits are formed by stamping or cutting the membrane to remove plastic film and form the shapes as indicated. Each of the fluidic circuits includes the flow channel 140 and the return channel 142. Some of the fluidic circuits illustrated in FIG. 23 include a mixing chamber, which may be symmetric (144a) or asymmetric (144b).

As shown in FIG. 23, the substrate 146 may include one or more target or capture zones 148. The substrate 146 is preferably made of polycarbonate and has a thin semi-reflective layer 151 deposited on the top thereof, seen in FIG. 24. This semi-reflective layer 151 is significantly thinner than the reflective layer 150 on the substrate 146 of FIG. 5. The thinner semi-reflective layer 151 allows for some transmission of the interrogation beam 119 through the structural layers of the transmissive disc as shown in FIG. 24.

The thin semi-reflective layer 151 may be formed from a metal such as aluminum or gold. Preferably, the thin semi-reflective layer 151 is from about 100 Angstroms to about 300 Angstroms in thickness and does not exceed 400 Angstroms. This thinner semi-reflective layer 151 allows a portion of the incident or interrogation beam 119 to penetrate and pass through the thin semi-reflective layer 151 to be detected by a top detector 184, shown in FIG. 27, while a portion of the light is reflected back. The relationship of the thickness of the thin semi-reflective layer 151 to its reflective and transmissive characteristics, when implemented with gold, are shown in Table 1. As indicated, the gold film layer is fully reflective at a thickness greater than 800 Angstroms, while the threshold density for transmission of light through the gold film is approximately 400 Angstroms.

TABLE 1
Gold Film Reflection and Transmission (Absolute Values)

| Thickness | Thickness | | |
|---|---|---|---|
| (Angstroms) | (nm) | Reflectance | Transmittance |
| 0 | 0 | 0.0505 | 0.9495 |
| 50 | 5 | 0.1683 | 0.7709 |
| 100 | 10 | 0.3981 | 0.5169 |
| 150 | 15 | 0.5873 | 0.3264 |
| 200 | 20 | 0.7142 | 0.2057 |
| 250 | 25 | 0.7959 | 0.1314 |
| 300 | 30 | 0.8488 | 0.0851 |
| 350 | 35 | 0.8836 | 0.0557 |
| 400 | 40 | 0.9067 | 0.0368 |
| 450 | 45 | 0.9222 | 0.0244 |
| 500 | 50 | 0.9328 | 0.0163 |
| 550 | 55 | 0.9399 | 0.0109 |
| 600 | 60 | 0.9448 | 0.0073 |
| 650 | 65 | 0.9482 | 0.0049 |
| 700 | 70 | 0.9505 | 0.0033 |
| 750 | 75 | 0.9520 | 0.0022 |
| 800 | 80 | 0.9531 | 0.0015 |

FIG. 25 is a top plan view of the optical bio-disc 110 illustrated in FIGS. 23 and 24 with the transparent cap portion 130 revealing the fluidic channels, the trigger markings 135, and the target zone 148 as situated within the disc.

An enlarged perspective view of the transmissive embodiment of an optical bio-disc 110 according to the present invention is shown in FIG. 26. The disc 110 is illustrated with a portion of the various layers thereof cut away to illustrate a partial sectional view of each principle, layer, substrate, coating, or membrane. FIG. 26 illustrates a transmissive disc format with the clear cap portion 130, the thin semi-reflective layer 151 on the substrate 146, and trigger markings 135. Trigger markings 135 include opaque material placed on the top portion of the cap, clear, non-reflective windows etched on the thin reflective layer 143 of the disc, or any mark that absorbs or does not reflect the signal coming from the trigger detector 186, shown in FIG. 27.

FIG. 26 also shows target zones 148 formed by marking the designated area in the indicated shape or alternatively in any desired shape. Markings to indicate target zones 148 may be made on the thin semi-reflective layer 151 on the substrate 146 or on the bottom portion of the substrate 146, under the disc. Preferably, target zones do not have physical boundaries but are known to the system by encoded addresses. Alternatively, the target zones 148 may be formed by a masking technique that includes masking the entire thin semi-reflective layer 151 except the target zones 148. In this embodiment, target zones 148 may be created by silk screening ink onto the thin semi-reflective layer 151.

An active layer 154 is applied over the thin semi-reflective layer 151. In the preferred embodiment, the active layer 154 may be formed from a modified polystyrene, for example, polystyrene-co-maleic anhydride. Alternatively, gold, activated glass, or modified glass may be used. As illustrated in this embodiment, the plastic adhesive member 136 is applied over the active layer 154. The exposed section of the plastic adhesive member 136 illustrates the cut out or stamped U-shaped form that creates the fluidic circuits 138. The final principle structural layer in this embodiment of the present bio-disc 110 is the clear, non-reflective cap portion 130 that includes the inlet 132 and vent port 134.

A schematic diagram illustrating the optical components 116 and light or laser source 118 that produces an incident or interrogation beam 119, a return beam 120, and a transmitted beam 180 is shown in FIG. 27. As discussed above, the incident or interrogation beam 119 is reflected from the reflective surface 156 of the cap portion 130 in a reflective bio-disc, directing the return beam 120 into a bottom detector 182. In this alternative transmissive embodiment of the present invention, the transmitted beam 180 is detected by a top detector 184, such as a photodetector, and analyzed for the presence of reporter beads.

FIG. 27 also shows a hardware trigger mechanism that includes the trigger markings 135 on the disc and a trigger detector 186. The hardware trigger mechanism, which may be used with both reflective bio-discs and transmissive bio-discs, is coupled to the processor 126 such that the processor only collects data when the interrogation beam 119 is on a respective target zone 148.

Alternatively, a software trigger may also be used with the transmissive bio-disc embodiment. The software trigger uses the bottom detector 182 to signal the processor 126 to collect data as soon as the interrogation beam 119 hits the edge of a respective target zone 148. FIG. 27 also illustrates a drive motor 122, a controller 124 for controlling the rotation of the optical bio-disc 110, the processor 126 and analyzer 128 used for processing either the return beam 120, in the case of reflective discs, or the reflected and transmitted beams 120 and 180, respectively, in the case of transmissive discs.

With reference to FIG. 28, a cross sectional view of a transmissive bio-disc 110 according to the present invention is shown, with the clear cap portion 130 and the thin semi-reflective layer 151 on the substrate 146. The semi-reflective layer may include encoded information in the form of pits and lands or wobble grooves. FIG. 28 also shows the active layer 154 applied over the thin semi-reflective layer 151. As discussed above, the thin semi-reflective layer 151 allows the incident or interrogation beam 119 to penetrate and pass through the disc to be detected by a top detector 184, while some of the light is reflected back along the same path as the incident beam but in the opposite direction. The reflected light or return beam 120 is used for tracking of the light source 150 along the grooves 152 on the disc, seen in FIG. 29. The thickness of the thin semi-reflective layer 151 is determined by the minimum amount of reflected light required by the disc reader to maintain its tracking ability. In the disc embodiment illustrated in FIG. 28, a defined target zone 148 may be created by direct markings made on the thin semi-reflective layer 151 on the substrate 146. These marking may be done using silk screening or any equivalent method.

FIG. 29 is a cross sectional view of the transmissive disc embodiment shown in FIG. 28. The substrate 146 in this embodiment includes a series of tracking grooves 152. These grooves are in the form of a spiral extending from near the center of the disc toward the outer edge. The grooves 152 are implemented so that the interrogation beam 119 may track along the spiral and retrieve encoded information therefrom. FIG. 29 also shows the active layer 154 applied over the thin semi-reflective layer 151. As further illustrated in FIG. 29, the plastic adhesive member 136 is applied over the active layer 154. Comparing the transmissive disc shown in FIG. 29 to the reflective disc shown in FIG. 6, it should be noted that the transmissive disc lacks the reflective surface 156 on the cap portion 130. Thus, when the cap 130 is applied to the plastic adhesive member 136, including the desired cut-out shapes, in the transmissive disc, the flow channel 140 is thereby formed and the incident beam 119 may pass therethrough substantially unreflected in the absence of reporter beads or the like.

FIG. 30 is a longitudinal cross-section of the disc shown in FIG. 29. The tracking grooves 152 are not shown as the section is cut along one of the grooves. A narrow flow channel 140, perpendicular to the grooves 152, is shown in cross-section.

An alternate embodiment of the transmissive optical bio-disc 110, which utilizes an open-face or open-disc format, is shown in FIG. 31. In this embodiment, the substrate 146 is implemented as a proximal layer relative to the interrogation beam 119. The thin semi-reflective layer 151, showing tracking grooves 152, is next provided as illustrated. The distal layer relative to the interrogation beam 119 is provided by the active layer 154.

In this embodiment, the capture DNA 158 may be inverted upward when the disc is loaded in the drive (shown in FIG. 1). The target DNA 160 and reporter beads 162 are brought into proximity with the capture DNA 158 by a variety of different methods which include, for example, depositing a test sample on the disc with a pipette. In this alternative embodiment, the target zones 148 are simply formed by the application of a small volume of capture DNA 158 solution to the active layer 154 to form clusters of capture DNA in desired locations. Reporter beads in a complex with target DNA and capture DNA are detected by changes in transmittance of the interrogatory beam 119, using a top detector 184.

A sectional view of the disc shown in FIG. 31, taken longitudinally along one of the tracking grooves 152, is shown in FIG. 32.

FIG. 33 is a sample display of test results illustrative of any of the test methods described above. The test results represented in FIG. 33 may be readily displayed on the monitor 114 shown in FIG. 1. The bio-disc 110 according to the present invention may include encoded software that interacts with the drive 112, the controller 124, the processor 126, and the analyzer 128 as shown in FIGS. 1 and 2. This interactive software is implemented to facilitate the methods described herein and the display of results as represented in FIG. 33.

In FIG. 33, the output data illustrated was collected using a reflective disc format with 8 target zones 148, each shown as a separate lane of the display. Each lane contains both numerical and graphical data regarding “event counts” during transmission of the interrogatory beam to the target zone represented by that lane. In the absence of reporter beads, interrogatory beam 119 is reflected back along the same path and is detected by bottom detector 184. When the interrogatory beam strikes a reporter bead, however, some portion of the light is scattered and thus is not reflected back along the same path. This is detected as a drop in the amplitude of the reflected light. Each time the amplitude drops below a threshold value, it is counted as an event. Thus, the number of events counted is directly related to the concentration of beads present in the target zone and thus to the concentration of target DNA.

The first two target zones were not used in this experiment, as shown by blank lanes 188 and 190 in FIG. 33. Target zone 3 contained no capture DNA and was used a negative control. Thus, although reporter beads and target DNA were added to target zone 3, no hybridization occurred and no reporter beads were retained in the zone, resulting in only 11 event counts, shown in lane 192. Target zone 4, containing single-stranded biotinylated DNA immobilized on the active layer 154, served as a positive control with the streptavidin coated reporter beads. As shown at 194, 204 and 206, this positive control yielded 2189 event counts. Target zones 5, 6, and 7 contained capture DNA 158 specific for various target DNA types, while target zone 8 contained a mixture of all three types of capture DNA. The results are shown at 196, 198, 200 and 202. The target DNA type showing the highest signal is shown at 196, with the signal shown graphically 208 and numerically 210.

Having generally described the invention, the same will be more readily understood through reference to the following examples, which are provided by way of illustration, and are not intended to be limiting of the present invention.

### Example 1

### Preparing Reporter Beads

A 10 μl aliquot of 1 μm NeutrAvidin coated Fluospheres from Molecular Probes (Eugene, Oregon) was diluted with 200 μl CDB (2% bovine serum albumin, 145 mM NaCl, 1 MM MgCl₂, 0.1 mM ZnCl, 50 mM Tris, pH 7.4, 0.05% NaN₃), mixed by vortexing, and centrifuged at 20,000 rpm for 5 minutes. The supernatant was discarded, and the beads resuspended in 200 μl CDB, with vortexing, followed by recentrifugation at 10,000 rpm for 5 minutes. The supernatant was once again discarded, followed by the addition of 200 μl CDB, vortexing, and a third round of centrifugation. The supernatant was again discarded, and the washed beads suspended in 100 μl of CDB.

### Example 2

### Applying Active Layer to Bio-disc Substrate

Poly(styrene-co-maleic anhydride) solution was prepared by dissolving 1.6 g PSMA pellets (Sigma, St. Louis, Mo.) in reagent grade toluene to a final volume of 80 ml.

A polycarbonate disc having a gold reflective layer, with target zones formed therein by photolithography, was placed on a “spin coater,” or modified centrifuge, with the reflective surface up. While rotating the disc on the spin coater, the reflective surface was cleaned with reagent grade alcohol. The spin coater was then set to the spin settings shown in Table 2:

TABLE 2

| | | | Acceleration |
|---|---|---|---|
| Step | Speed | Time | Time |
| Spin 1 | 2500 rpm | 5 sec | 0.5 sec |
| Spin 2 | 4000 rpm | 5 sec | 10 sec |

A steady stream of PSMA solution was applied to the reflective surface of the disc as it accelerated from 2500 rpm to 4000 rpm, starting at the outer edge of the disc and moving to the center of the disc in one smooth stroke.

### Example 3

### Preparing the Bio-disc.

The disc from Example 2 was placed on a CD assembler/spindle with the active layer up. A clear cover with inlet and vent ports was placed on top of the disc and the disc was marked with a fine tip marker to indicate positions of the inlet and vent ports using the clear cover disc as a guide.

Capture DNA was applied to the target zones on the disc from Example 2 by immediately applying from about 1.6 μl to about 2.0 μl of 1 μM aminated oligonucleotides in PBS (150 mM NaCl, 50 mM phosphate buffer, pH 7.5) to the active layer at each target zone.

The disc was placed in a CD jewel case and incubated for 15 minutes at room temperature in a humid environment formed by placing water droplets on each corner of the closed CD jewel case, to allow the aminated oligonucleotides to bind to the active layer.

The disc was placed in a petri dish, washed twice in wash buffer (100 mM KCl or NaCl, 50 mM Tris, pH 7.4), and spin dried at 4000-5000 rpm.

A circular piece of plastic double-sided adhesive film with paper backing on one side was placed on the disc with the paper side up. The plastic adhesive film was pre-cut to match the size of the disc and included pre-cut fluidic circuits. The plastic adhesive film was placed onto the disc such that the marked inlet and vent ports were properly aligned with the fluidic circuits. Pressure was used to adhere the plastic adhesive to the disc, and the paper backing was removed.

A cap portion with a gold reflective surface and inlet and vent ports was applied to the disc, reflective surface down, with the inlet and vent ports aligned with the markings made above. Pressure was used to adhere the cap to the plastic adhesive film on the disc.

### Example 4

### General Bead Based DNA Assay

DNA blocking solution (5×Denhardt's solution, 0.1 mg/ml salmon sperm DNA, 200 mM KC1, 10 mM MgCl₂, 50 mM Tris, pH 7.4) was degassed in a vacuum desiccator and injected into the fluidic circuits of a bio-disc prepared as in Example 3 via the inlet port, taking care that no air bubbles remained in the circuits. The bio-disc was then incubated at room temperature for 30 to 60 minutes.

The DNA blocking solution was removed, and the fluidic circuits washed with hybridization buffer (200 mM NaCl, 10 mM MgCl₂, 50 mM Tris, pH 7.4) injected into the inlet ports using a syringe. PCR amplicons were diluted 1:2 in 2×hybridization buffer, denatured at 95° C. for 5 minutes and immediately placed on ice for 5 minutes.

The denatured amplicons were added to the appropriate fluidic circuits via the inlet port (10 μl per fluidic circuit) and allowed to hybridize for 1.5 to 2 hours at room temperature.

Following hybridization, the fluidic circuits were washed with hybridization buffer injected with a syringe into the inlet ports. Reporter beads, prepared as in Example 1, were then added to each fluidic circuit (10 μl suspended beads per fluidic circuit). Each fluidic circuit was sealed with tape, and the bio-disc was spun at 1500 rpm for 15 minutes, followed by 4500 rpm for 10 minutes. The bio-disc was then placed in a disc-reader, similar to that shown in FIG. 2, and scanned with a 780 nm lightbeam, with the light reflected from the bio-disc at each target zone measured to detect changes in the amplitude of the reflected light.

### Example 5

### Bead Based DNA Assay Used to Identify Brucella Strains

A bio-disc with 8 target zones was prepared as in Example 3, with 2.0 μl of 1 μM aminated DNA oligonucleotides specific to one of the Brucella strains applied to each target zone, as indicated in Table 3, below.

Brucella sp. genomic DNA was subjected to PCR amplification using three different forward/reverse primer sets, each set specific to one of three Brucella strains, as well as to a multiplexed PCR amplification using all three primer sets, as indicated in Table 3, to generate three different amplicons. Each reaction contained 1 ng/μl Brucella DNA, 0.2 μM biotinylated forward and reverse primers, 0.2 mM dNTPs, 0.05 U/μl Taq polymerase, 3.0 mM MgCl₂ and 1×PCR buffer. The thermocycle conditions were:

Step 1: 95° C. for 12.5 minutes

Step 2: 95° C. for 0.5 minutes

Step 3: 54° C. for 0.5 minutes

Step 4: 72° C. for 0.5 minutes

Step 5: Repeat Steps 2-4 34 times

Step 6: 72° C. for 5.0 minutes

The bead based DNA assay was performed as described in Example 4, with the results indicated in Table 3. As used in Table 3, “Multiplex” refers to a mixed primer set with forward and reverse primers directed to all three Brucella strains.

TABLE 3

| | | Biotinylated | Event |
|---|---|---|---|
| Target Zone | Capture DNA | PCR primers | Counts |
| 1 Not used | None | None | N/A |
| 2 Not used | None | None | N/A |
| 3 Control (−) | None | None | 58 |
| 4 Test 1 | B. abortus | Multiplex | 706 |
| 5 Test 2 | B. melitensis | Multiplex | 388 |
| 6 Test 3 | B. suis | Multiplex | 657 |
| 7 Test 4 | Mix of Brucella | Multiplex | 824 |
| | strains | | |
| 8 Control (+) | Biotinylated | None | 1446 |
| | DNA | | |

As shown in Table 3, the bead base DNA assay suggests the presence of all three Brucella strains in the sample, with B. abortus generating the highest signal.

### Example 6

### Bead Based DNA Assay Using Signal DNA Pre-coupled to Beads

NeutrAvidin labeled microspheres (FluorSpheres; Molecular Probes) were prepared as described in Example 1. Final bead suspensions contained 1% solids or 150 μg/15 μl. The biotin binding capacity of the microspheres was 4.4 nmoles biotin/mg microspheres, or 6.6×10⁻¹⁰ moles biotin/15 μl suspension, which represents 3.96×10¹⁴ biotin binding sites/15 μl bead suspension.

A 3 μl aliquot of a 244 μM stock solution of biotinylated signal DNA oligonucleotide (7.32×10⁻¹⁰ moles or 4.4×10¹⁴ molecules) was added to 15 μl of the bead suspension, resulting in approximately 1 signal DNA probe per biotin binding site. The beads and signal DNA probes were incubated for 2 hours at room temperature with continuous mixing. Following the incubation, the beads were washed 3 times in CDB buffer to remove non-incorporated signal DNA.

A bio-disc was prepared as in Example 3, except that 1 μl of 1 μM capture DNA was applied to each target zone, followed by a 1 hour incubation at room temperature. Degassed DNA blocking solution (5×Denhardt's solution, 0.1 mg/ml salmon sperm DNA, 200 mM KCl, 10 mM MgCl₂, 50 mM Tris, pH 7.4) was injected into the fluidic circuits of the bio-disc, which was then incubated at room temperature for 30 to 40 minutes. The DNA blocking solution was removed, and the fluidic circuits washed with hybridization buffer (200 mM NaCl, 10 mM MgCl₂, 50 mM TrisCl, pH 7.4)

A 1 μM solution of target DNA (single stranded DNA, 60 to 80 nucleotides in length, with areas of sequence complementary to the capture DNA and to the signal DNA, in hybridization buffer) was added to the bio-disc and incubated for 0.5 to 1 hour at room temperature. The target DNA solution was removed, the fluidic circuits were filled with beads coupled to signal DNA, and the bio-disc was incubated for 1 hour at 37° C.

Unbound reporter beads were removed by three successive spins: <2000 rpm for 5 minutes, followed by two 5000 rpm spins for 5 minutes each. Bead binding was observed and measured using a FluorImager (Molecular Dynamics). The results are shown in Table 4.

TABLE 4

| | Fluorescence Signal |
|---|---|
| | (arbitrary units) (average of |
| Target | 4 capture zones) |
| Target 1 | 19602 |
| Target 2 | 38891 |
| Negative control | 43 |
| Positive control | 94867 |

### Example 7

### Bead Based Bridging Hybridization with Nopalin Synthetase Terminator (NosT) Amplicon

A 10 μl aliquot of a 1 μM suspension of NeutrAvidin labeled microspheres (FluorSpheres; Molecular Probes; 1.8×10¹⁰ particles/ml) were prepared as described in Example 1. Final bead suspensions contained 1% solids or 100 μg/10 μl. The biotin binding capacity of the microspheres was 4.4 nmoles biotin/mg microspheres, or 4.4×10⁻¹⁰ moles biotin/10 μl suspension, which represents 2.65×10¹⁴ biotin binding sites/10 μl bead suspension.

A 2 μl aliquot of a 260 μM stock solution of biotinylated signal DNA oligonucleotide (5.2×10⁻¹⁰ moles or 3.13×10¹⁴ molecules) having a 44 base stretch of sequence complementary to a portion of a NosT amplicon was added to 10 μl of the bead suspension, resulting in approximately 1.2 signal DNA probes per biotin binding site. The beads and signal DNA probes were incubated for 1 hour at room temperature with continuous mixing. Following the incubation, the beads were washed 3 times in CDB buffer to remove non-incorporated signal DNA and reconstituted in 100 μl CDB buffer.

20 μl of PCR amplified NosT target DNA amplicon was heat denatured for 5 minutes at 95° C., added to the reporter beads/signal DNA suspension, and incubated for 2 hours at room temperature with mixing. The beads/signal DNA/target DNA complexes were washed three times in CDB buffer, and reconstituted in 40 μl CDB buffer.

A bio-disc was prepared as in Example 3, with 1 μM capture DNA (aminated NosT oligonucleotide probe, with a 31 base stretch of sequence complementary to a portion of the NosT amplicon) solution applied to each target zone, followed by a 1 hour incubation at room temperature. Degassed DNA blocking solution (5×Denhardt's solution, 0.1 mg/ml salmon sperm DNA, 200 mM KCl, 10 mM MgCl₂, 50 mM Tris, pH 7.4) was injected into the fluidic circuits of the bio-disc, which was then incubated at room temperature for 30 to 40 minutes. The DNA blocking solution was removed, and the fluidic circuits washed with hybridization buffer (200 mM NaCl, 10 MM MgCl₂, 50 mM TrisCl, pH 7.4) Reporter bead/signal DNA/target DNA complex solution was added to the fluidic circuits and allowed to hybridize for 2 hours at room temperature. The bio-disc was then spun three times at 4500 rpm for 10 minutes to remove unbound beads. Bead binding was observed and measured using a FluorImager (Molecular Dynamics). The results are shown in Table 5.

TABLE 5

| Target | Bead Fluorescence (N = 3) |
|---|---|
| Target 1 | 23699 |
| Negative Control | 2321 |
| Positive Control | 785594 |

All patents, patent applications, and other publications mentioned in this specification are incorporated herein in their entireties by reference.

While this invention has been described in detail with reference to a certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

What is claimed is:

1. An optical bio-disc, comprising:
a substantially circular substrate having a center and an outer edge;
an active layer associated with said substrate; and
a strand of DNA including a reactive group which has an affinity for said active layer so that said reactive group attaches to said active layer to immobilize said strand of DNA in a target zone disposed between said center and said outer edge.

2. The optical bio-disc according to claim 1 wherein said strand of DNA is a single strand of DNA.

3. The optical bio-disc according to claim 1 wherein said strand of DNA includes a double strand of DNA.

4. The optical bio-disc according to any one of claims 1, 2, or 3 wherein said active layer is formed from a modified polystyrene.

5. The optical bio-disc according to claim 4 wherein said modified polystyrene is polystyrene-co-maleic anhydride.

6. A surface assembly for immobilizing DNA capture probes, said assembly comprising:
a substrate;
an active layer associated with said substrate; and
a strand of DNA including a reactive group which has an affinity for said active layer so that said reactive group attaches to said active layer to thereby immobilize said strand of DNA.

7. The assembly according to claim 6 wherein said strand of DNA is a single strand of DNA.

8. The assembly according to claim 6 wherein said strand of DNA includes a double strand of DNA.

9. The assembly according to any one of claims 6, 7, or 8 wherein said active layer is formed from a modified polystyrene.

10. The assembly according to claim 9 wherein said modified polystyrene is polystyrene-co-maleic anhydride.

11. An optical bio-disc, comprising:
a substrate having a tracking groove formed therein;
a reflective layer formed on at least a portion of said substrate so that an incident beam of electromagnetic energy may track along said groove
an active layer associated with said substrate; and
a strand of DNA including a reactive group which has an affinity for said active layer so that said reactive group attaches to said active layer to immobilize said strand of DNA.

12. The optical bio-disc according to claim 11 wherein said strand of DNA is a single strand of DNA.

13. The optical bio-disc according to claim 11 wherein said strand of DNA includes a double strand of DNA.

14. The optical bio-disc according to any one of claims 11, 12, or 13 wherein said active layer is formed from a modified polystyrene.

15. The optical bio-disc according to claim 14 wherein said modified polystyrene is polystyrene-co-maleic anhydride.

16. An optical bio-disc, comprising:
a substantially circular substrate having a center and an outer edge;
an active layer associated with said substrate; and
a strand of DNA including an amino group which has an affinity for said active layer so that said amino group attaches to said active layer to immobilize said strand of DNA in a target zone disposed between said center and said outer edge.

17. The optical bio-disc according to claim 16 wherein said strand of DNA is a single strand of DNA.

18. The optical bio-disc according to claim 16 wherein said strand of DNA includes a double strand of DNA.

19. The optical bio-disc according to any one of claims 16, 17, or 18 wherein said active layer is formed from a modified polystyrene.

20. The optical bio-disc according to claim 19 wherein said modified polystyrene is polystyrene-co-maleic anhydride.

21. An optical bio-disc, comprising:
a substrate having encoded information associated therewith, said encoded information being readable by a disc drive assembly to control rotation of the disc;
a target zone associated with said substrate, said target zone disposed at a predetermined location relative to said substrate
an active layer associated with said target zone; and
a strand of DNA including a reactive group which attaches to said active layer so that when said substrate is rotated, said reactive group remains attached to said active layer to thereby maintain a number of said strands of DNA within said target zone.

22. The optical bio-disc according to claim 21 wherein said strand of DNA is a single strand of DNA.

23. The optical bio-disc according to claim 21 wherein said strand of DNA includes a double strand of DNA.

24. The optical bio-disc according to any one of claims 21, 22, or 23 wherein said active layer is formed from a modified polystyrene.

25. The optical bio-disc according to claim 23 wherein said modified polystyrene is polystyrene-co-maleic anhydride.

26. An optical bio-disc for testing for the presence of a target-DNA in a DNA sample, said bio-disc comprising:
a substrate having a center and an outer edge, and having encoded information associated therewith, said encoded information being readable by a disc drive assembly to control rotation of the disc;
a target zone associated with said substrate, said target zone disposed at a predetermined location relative to said center of said substrate;
an active layer associated with said target zone;
a strand of capture-DNA including a reactive group that attaches to said active layer to immobilize said strand of capture-DNA within said target zone;
a flow channel in fluid communication with said target zone;
a plurality of reporters deposited in said flow channel, each of said reporters having attached thereto a plurality of strands of signal-DNA, said capture-DNA and said signal-DNA being non-complementary; and
an input site in fluid communication with said flow channel, said input site implemented to receive a DNA sample to be tested for the presence of a target-DNA that is complementary to said capture-DNA and said signal-DNA, so that when said DNA sample is deposited in said flow channel, said DNA sample and said reporters move into said target zone and hybridization occurs between said target-DNA and said capture-DNA, and said target-DNA and said signal-DNA thereby placing said reporters in said target zone when target-DNA is present in the DNA sample.

27. An optical bio-disc for testing for the presence of a target-DNA in a DNA sample, said bio-disc comprising:
a substrate having a center and encoded information associated therewith, said encoded information being readable by a disc drive assembly to control rotation of the disc;
a target zone associated with said substrate, said target zone disposed at a predetermined location relative to said center of said substrate;
an active layer associated with said target zone;
a strand of capture-DNA including a reactive group that attaches to said active layer to immobilize said strand of capture-DNA within said target zone;
a flow channel in fluid communication with said target zone;
a plurality of reporters deposited in said flow channel, each of said reporters including a binder that has an affinity for a target-DNA that is complementary to said capture-DNA; and
an input site in fluid communication with said flow channel, said input site implemented to receive a DNA sample to be tested for the presence of said target-DNA, so that when said DNA sample is deposited in said flow channel, said DNA sample and said reporters move into said target zone and hybridization occurs between said target-DNA and said capture-DNA to thereby place said reporters in said target zone when target-DNA is present in the DNA sample.

28. An optical bio-disc for determining the presence of a target-DNA in a test sample, said bio-disc comprising:
a substrate having a center and an outer edge, and having encoded information associated therewith, said encoded information being readable by a disc drive assembly to control rotation of the disc;
a target zone associated with said substrate, said target zone disposed at a predetermined location relative to said center of said substrate;
an active layer associated with said target zone;
a strand of capture-DNA including a reactive group that attaches to said active layer to immobilize said strand of capture-DNA within said target zone;
a flow channel in fluid communication with said target zone; and
an input site in fluid communication with said flow channel, said input site implemented to receive a test sample including sample-DNA and a plurality of reporters, each of said reporters having attached thereto a plurality of strands of signal-DNA, said capture-DNA and said signal-DNA being non-complementary, said sample-DNA to be tested for the presence of a target-DNA that is complementary to said capture-DNA and said signal-DNA so that when said test sample is deposited in said flow channel, said test sample moves into said target zone and hybridization occurs between any target-DNA and said capture-DNA to thereby maintain said reporters in said target zone when target-DNA is present in the sample-DNA.

29. An optical bio-disc for determining the presence of a target-DNA in a test sample, said bio-disc comprising:
a substrate having a center and encoded information associated therewith, said encoded information being readable by a disc drive assembly to control rotation of the disc;
a target zone associated with said substrate, said target zone disposed at a predetermined location relative to said center of said substrate;
an active layer associated with said target zone;
a strand of capture-DNA including a reactive group that attaches to said active layer to immobilize said strand of capture-DNA within said target zone;
a flow channel in fluid communication with said target zone; and
an input site in fluid communication with said flow channel, said input site implemented to receive a test sample including sample-DNA and a plurality of reporters, each of said reporters including a binder that has an affinity for a target-DNA that is complementary to said capture-DNA, said sample-DNA to be tested for the presence of said target-DNA so that when said test sample is deposited in said flow channel, said test sample and said reporters move into said target zone and hybridization occurs between any target-DNA and said capture-DNA to thereby maintain said reporters in said target zone when target-DNA is present in the sample-DNA.

30. The optical bio-disc according to any one of claims 26, 27, 28, or 29 wherein the presence of said target-DNA is determined by directing a beam of electromagnetic energy from the disc drive assembly toward said target zone and analyzing electromagnetic energy returned from said reporters.

31. A method of testing for the presence of a target-DNA in a DNA sample by use of an optical bio-disc, said method comprising the steps of:
preparing a DNA sample to be tested for the presence of a target-DNA;
preparing a plurality of reporters each having attached thereto a plurality of strands of signal-DNA, the target-DNA and signal-DNA being complementary;
mixing said DNA sample and said plurality of reporters to thereby form a test sample;
allowing hybridization between said signal-DNA and any target-DNA existing in the DNA sample;
depositing said test sample in a flow channel of an optical bio-disc which is in fluid communication with a target zone, the target zone including a plurality of strands of capture-DNA each including a reactive group that attaches to an active layer to immobilize the strands of capture-DNA within the target zone, the capture-DNA and the signal-DNA being non-complementary;
allowing any target-DNA to hybridize with the capture-DNA so that reporters associated with the target-DNA are maintained within the capture zone;
removing from the target zone reporters with signal-DNA that is free of any target-DNA; and
detecting any reporters in the target zone to thereby determine whether target-DNA is present in the DNA sample.

32. A method of testing for the presence of a target-DNA in a DNA sample by use of an optical bio-disc, said method comprising the steps of:
preparing a DNA sample to be tested for the presence of a target-DNA;
depositing said DNA sample in a mixing chamber of an optical bio-disc which is linked to a target zone by a connecting flow channel, the mixing chamber including a plurality of reporters, each of said reporters including a binder that has an affinity for the target-DNA;
allowing any target-DNA existing in the DNA sample to bind to the reporters;
rotating the optical bio-disc to cause the DNA sample to move from the mixing chamber through the flow channel and into the target zone, the target zone including a plurality of strands of capture-DNA each including a reactive group that attaches to an active layer to immobilize the strands of capture-DNA within the target zone, the capture-DNA and the target-DNA being complementary;
allowing any target-DNA to hybridize with the capture-DNA so that reporters associated with the target-DNA are maintained within the capture zone;
removing from the target zone reporters that are free of any target-DNA; and
detecting any reporters in the target zone to thereby determine whether target-DNA is present in the DNA sample.

33. A method of testing for the presence of a target-DNA in a DNA sample by use of an optical bio-disc, said method comprising the steps of:
preparing a DNA sample to be tested for the presence of a target-DNA;
depositing said DNA sample in a mixing chamber of an optical bio-disc which is linked to an interconnecting flow channel including a target zone, the mixing chamber including a plurality of reporters each having attached thereto a plurality of strands of signal-DNA, the target-DNA and signal-DNA being complementary;
allowing hybridization between said signal-DNA and any target-DNA existing in the DNA sample while in the mixing chamber of the optical bio disc;
rotating the optical bio-disc to cause the DNA sample to move from the mixing chamber through the flow channel and into the target zone, the target zone including a plurality of strands of capture-DNA each including a reactive group that attaches to an active layer to immobilize the strands of capture-DNA within the target zone, the capture-DNA and the signal-DNA being non-complementary;
allowing any target-DNA to hybridize with the capture-DNA so that reporters associated with the target-DNA are maintained within the capture zone;
removing from the target zone reporters with signal-DNA that is free of any target-DNA; and
detecting any reporters in the target zone to thereby determine whether target-DNA is present in the DNA sample.

34. A method of testing for the presence of a target-DNA in a DNA sample by use of an optical bio-disc, said method comprising the steps of:
preparing a DNA sample to be tested for the presence of a target-DNA;
preparing a plurality of reporters each of said reporters including a binder that has an affinity for the target-DNA;
mixing said DNA sample and said plurality of reporters to thereby form a test sample;
allowing any target-DNA existing in the DNA sample to attach to the reporters;
depositing said test sample in a flow channel of an optical bio-disc which is in fluid communication with a target zone, the target zone including a plurality of strands of capture-DNA each including a reactive group that attaches to an active layer to immobilize the strands of capture-DNA within the target zone, the capture-DNA and the target-DNA being complementary;
allowing any target-DNA to hybridize with the capture-DNA so that reporters associated with the target-DNA are maintained within the capture zone;
removing from the target zone reporters that are free of any target-DNA; and
detecting any reporters in the target zone to thereby determine whether target-DNA is present in the DNA sample.

35. The method according to either claim 31, 32, 33 or 34 wherein said removing step is performed by rotating the optical bio-disc.

36. The method according to any one of claims 31, 32, 33, or 34 wherein the presence of said target-DNA is determined by directing a beam of electromagnetic energy from a disc drive assembly toward said target zone and analyzing electromagnetic energy reflected from said bio-disc.

37. The method according to any one of claims 31, 32, 33, or 34 wherein the presence of said target-DNA is determined by directing a beam of electromagnetic energy from a disc drive assembly toward said target zone and analyzing electromagnetic energy transmitted through said bio-disc.

38. A method of making an optical bio-disc for testing for the presence of a target-DNA in a DNA sample, said method comprising the steps of:
providing a substrate having a center and an outer edge;
encoding information on an information layer associated with the substrate, said encoded information being readable by a disc drive assembly to control rotation of the disc;
forming a target zone in association with said substrate, said target zone disposed at a predetermined location relative to said center of said substrate;
applying an active layer in said target zone;
depositing within said target zone, a plurality of strands of capture-DNA each including a reactive group that attaches to said active layer to immobilize said strands of capture-DNA within said target zone
forming a flow channel in fluid communication with said target zone;
forming a mixing chamber in fluid communication with the flow channel;
depositing a plurality of reporters in the mixing chamber, each of said reporters including a binder that has an affinity for a target-DNA that is complementary to said capture-DNA; and
designating an input site associated with the mixing chamber, the input site implemented to receive a DNA sample to be tested for the presence of any target-DNA, so that when the DNA sample is deposited in the mixing chamber and the disc is rotated, the DNA sample and the reporters move into the target zone and hybridization occurs between the target-DNA and the capture-DNA to thereby place the reporters in the target zone when target-DNA is present in the DNA sample.

39. A method of making an optical bio-disc for determining the presence of a target-DNA in a test sample, said method comprising the steps of:
providing a substrate having a center and an outer edge;
encoding information on an information layer associated with the substrate, the encoded information being readable by a disc drive assembly to control rotation of the disc;
forming a target zone in association with the substrate, the target zone disposed at a predetermined location relative to the center of the substrate;
applying an active layer in the target zone;
depositing within the target zone, a plurality of strands of capture-DNA each including a reactive group that attaches to the active layer to immobilize the strands of capture-DNA within the target zone; and
forming a flow channel in fluid communication with the target zone, the flow channel implemented to receive a test sample including sample-DNA and a plurality of reporters, each of the reporters having attached thereto a plurality of strands of signal-DNA, the capture-DNA and the signal-DNA being non-complementary, the sample-DNA to be tested for the presence of a target-DNA that is complementary to the capture-DNA and the signal-DNA so that when the test sample is deposited in the flow channel, the test sample moves into the target zone and hybridization occurs between any target-DNA and the capture-DNA to thereby maintain reporters with hybridized DNA in the target zone when target-DNA is present in the sample-DNA.

40. A method of making an optical bio-disc for determining the presence of a target-DNA in a test sample, said method comprising the steps of:
providing a substrate having a center;
encoding information on an information layer associated with the substrate, the encoded information being readable by a disc drive assembly to control rotation of the disc;
forming a target zone in association with the substrate, the target zone disposed at a predetermined location relative to the center of the substrate;
depositing an active layer in the target zone;
depositing in the target zone, a plurality of strands of capture-DNA each including a reactive group that attaches to the active layer to immobilize the strands of capture-DNA within the target zone; and
forming a flow channel in fluid communication with the target zone, the flow channel implemented to receive a test sample including sample-DNA and a plurality of reporters, each of the reporters including a binder that has an affinity for a target-DNA that is complementary to the capture-DNA, the sample-DNA to be tested for the presence of the target-DNA so that when the test sample is deposited in the flow channel, the test sample and the reporters move into the target zone and hybridization occurs between any target-DNA and the capture-DNA to thereby maintain the reporters in the target zone when target-DNA is present in the sample-DNA.

41. A method of making an optical bio-disc to test for the presence of a target-DNA in a DNA sample, the method comprising the steps of:
providing a substrate having a center and an outer edge;
encoding information on an information layer associated with the substrate, the encoded information being readable by a disc drive assembly to control rotation of the disc;
forming a target zone in association with the substrate, the target zone disposed at a predetermined location relative to the center of the substrate;
depositing an active layer in the target zone;
depositing a plurality of strands of capture-DNA in the target zone, each strand of capture-DNA including a reactive group that attaches to the active layer to immobilize the strands of capture-DNA within the target zone;
forming a flow channel in fluid communication with the target zone;
forming a mixing chamber in fluid communication with the flow channel; and
depositing a plurality of reporters in the mixing chamber, each of the reporters having attached thereto a plurality of strands of signal-DNA, the capture-DNA and the signal-DNA being non-complementary.

42. The method according to any one of claims 38, 39, 40 or 41 wherein the presence of the target-DNA is determined by directing a beam of electromagnetic energy from the disc drive assembly toward the target zone and analyzing electromagnetic energy returned from the reporters.

43. An optical bio-disc, comprising:
a substrate having a center and an outer edge, said substrate forming a distal layer of the bio-disc, said substrate having a top surface and a bottom surface relative to an interrogation beam of electromagnetic energy directed from a disc drive;
a reflective layer formed on the bottom surface of said substrate;
an active layer associated with said substrate and said reflective layer; and
a strand of capture DNA including a reactive group which has an affinity for said active layer so that said reactive group attaches to said active layer to immobilize said strand of DNA in a target zone disposed between said center and said outer edge.

44. The optical bio-disc according to claim 43 wherein said strand of capture DNA is complementary to a strand of target DNA which includes a reporter that is detectable by said interrogation beam.

45. The optical bio-disc according to either claim 43 or 44 wherein said strand of capture DNA is a single strand of DNA.

46. The optical bio-disc according to either claim 43 or 44 wherein said strand of capture DNA includes a double strand of DNA.

47. The optical bio-disc according to either claim 43 or 44 wherein said active layer is formed from a modified polystyrene.

48. The optical bio-disc according to claim 47 wherein said modified polystyrene is polystyrene-co-maleic anhydride.

49. The optical bio-disc according to either claim 43 or 44 wherein said reflective layer is interposed between said substrate and said active layer.

50. A method of testing for the presence of a target-RNA in a sample by use of an optical bio-disc, said method comprising the steps of:
preparing a sample to be tested for the presence of a target-RNA;
preparing a plurality of reporters each having attached thereto a plurality of strands of signal-DNA, the target-RNA and signal-DNA being complementary;
mixing said sample and said plurality of reporters to thereby form a test sample;
allowing hybridization between said signal-DNA and any target-RNA existing in the DNA sample;
depositing said test sample in a flow channel of an optical bio-disc which is in fluid communication with a target zone, the target zone including a plurality of strands of capture-DNA each including a reactive group that attaches to an active layer to immobilize the strands of capture-DNA within the target zone, the capture-DNA and the signal-DNA being non-complementary;
allowing any target-RNA to hybridize with the capture-DNA so that reporters associated with the target-RNA are maintained within the capture zone;
removing from the target zone reporters with signal-DNA that is free of any target-RNA; and
detecting any reporters in the target zone to thereby determine whether target-RNA is present in the sample.

51. A method of testing for the presence of a target-RNA in a sample by use of an optical bio-disc, said method comprising the steps of:
preparing a sample to be tested for the presence of a target-RNA;
depositing said sample in a mixing chamber of an optical bio-disc which is linked to a target zone by a connecting flow channel, the mixing chamber including a plurality of reporters, each of said reporters including a binder that has an affinity for the target-RNA;
allowing any target-RNA existing in the sample to bind to the reporters;
rotating the optical bio-disc to cause the sample to move from the mixing chamber through the flow channel and into the target zone, the target zone including a plurality of strands of capture-DNA each including a reactive group that attaches to an active layer to immobilize the strands of capture-DNA within the target zone, the capture-DNA and the target-RNA being complementary;
allowing any target-RNA to hybridize with the capture-DNA so that reporters associated with the target-RNA are maintained within the capture zone;
removing from the target zone reporters that are free of any target-RNA; and
detecting any reporters in the target zone to thereby determine whether target-RNA is present in the sample.

52. A method of testing for the presence of a target-RNA in a sample by use of an optical bio-disc, said method comprising the steps of:
preparing a sample to be tested for the presence of a target-RNA;
depositing said sample in a mixing chamber of an optical bio-disc which is linked to an interconnecting flow channel including a target zone, the mixing chamber including a plurality of reporters each having attached thereto a plurality of strands of signal-DNA, the target-RNA and signal-DNA being complementary;
allowing hybridization between said signal-DNA and any target-RNA existing in the sample while in the mixing chamber of the optical bio disc;
rotating the optical bio-disc to cause the sample to move from the mixing chamber through the flow channel and into the target zone, the target zone including a plurality of strands of capture-DNA each including a reactive group that attaches to an active layer to immobilize the strands of capture-DNA within the target zone, the capture-DNA and the signal-DNA being non-complementary;
allowing any target-RNA to hybridize with the capture-DNA so that reporters associated with the target-RNA are maintained within the capture zone;
removing from the target zone reporters with signal-DNA that is free of any target-RNA; and
detecting any reporters in the target zone to thereby determine whether target-RNA is present in the DNA sample.

53. A method of testing for the presence of a target-RNA in a sample by use of an optical bio-disc, said method comprising the steps of:
preparing a sample to be tested for the presence of a target-RNA;
preparing a plurality of reporters, each of said reporters including a binder that has an affinity for the target-RNA; mixing said sample and said plurality of reporters to thereby form a test sample;
allowing any target-RNA existing in the sample to attach to the reporters;
depositing said test sample in a flow channel of an optical bio-disc which is in fluid communication with a target zone, the target zone including a plurality of strands of capture-DNA each including a reactive group that attaches to an active layer to immobilize the strands of capture-DNA within the target zone, the capture-DNA and the target-RNA being complementary;
allowing any target-RNA to hybridize with the capture-DNA so that reporters associated with the target-RNA are maintained within the capture zone;
removing from the target zone reporters that are free of any target-RNA; and
detecting any reporters in the target zone to thereby determine whether target-RNA is present in the sample.

54. The method according to either claim 50, 51, 52 or 53 wherein said removing step is performed by rotating the optical bio-disc.

55. The method according to any one of claims 50, 51, 52, or 53 wherein the presence of said target-RNA is determined by directing a beam of electromagnetic energy from a disc drive assembly toward said target zone and analyzing electromagnetic energy reflected from said bio-disc.

56. The method according to any one of claims 50, 51, 52, or 53 wherein the presence of said target-RNA is determined by directing a beam of electromagnetic energy from a disc drive assembly toward said target zone and analyzing electromagnetic energy transmitted through said bio-disc.

57. A method of testing for the presence of a target-DNA in a DNA sample by use of an optical bio-disc, said method comprising the steps of:
preparing a DNA sample to be tested for the presence of a target-DNA, said DNA sample including an affinity agent;
preparing a plurality of reporters each of said reporters including a plurality of binders that has an affinity for the affinity agent on the target-DNA;
depositing said DNA sample in a flow channel of an optical bio-disc which is in fluid communication with a target zone, the target zone including at least one strand of capture-DNA that is attached to an active layer to immobilize the strands of capture-DNA within the target zone, the capture-DNA being complementary to a segment of the target-DNA;
allowing any target-DNA to hybridize with the capture-DNA; removing from the target zone unbound DNA sample; depositing said reporters in said flow channel;
allowing reporters to bind to the bound target-DNA on said target zone so that reporters associated with the target-DNA are maintained within the capture zone;
removing from the target zone unbound reporters; and
detecting reporters in the target zone to thereby determine whether target-DNA is present in the DNA sample.

58. A method of testing for the presence of a target-DNA in a DNA sample by use of an optical bio-disc, said method comprising the steps of:
preparing a DNA sample to be tested for the presence of a target-DNA;
preparing a plurality of signal-DNA for association with said target-DNA, the target-DNA and signal-DNA being complimentary at least in part, said signal-DNA including an affinity agent;
preparing a plurality of reporters each of said reporters including a plurality of binders that has an affinity for the affinity agent on the signal-DNA;
depositing said DNA sample in a flow channel of an optical bio-disc which is in fluid communication with a target zone, the target zone including a plurality of strands of capture-DNA that attach to an active layer to immobilize the strands of capture-DNA within the target zone, the capture-DNA and the target-DNA being at least partially complementary;
allowing any target-DNA to hybridize with the capture-DNA; removing from the target zone unbound DNA sample; depositing said signal-DNA in said flow channel;
allowing signal-DNA to hybridize to said target-DNA in the target zone, said signal-DNA and said capture-DNA being non-complimentary;
removing from the target zone unbound signal-DNA;
depositing said reporters in said flow channel;
allowing reporters to bind to the bound signal-DNA so that reporters associated with the target-DNA are maintained within the capture zone;
removing from the target zone unbound reporters; and
detecting reporters in the target zone to thereby determine whether target-DNA is present in the DNA sample.

59. A method of testing for the presence of a target-RNA in an RNA sample by use of an optical bio-disc, said method comprising the steps of:
preparing an RNA sample to be tested for the presence of a target-RNA;
preparing a plurality of signal-DNA for association with said target-RNA, the target-RNA and signal-DNA being complimentary at least in part, said signal-DNA including an affinity agent;
preparing a plurality of reporters each of said reporters including a plurality of binders that has an affinity for the affinity agent on the signal-DNA;
depositing said RNA sample in a flow channel of an optical bio-disc which is in fluid communication with a target zone, the target zone including a plurality of strands of capture-DNA that attach to an active layer to immobilize the strands of capture-DNA within the target zone, the capture-DNA and the target-RNA being at least partially complementary;
allowing any target-RNA to hybridize with the capture-DNA;
removing from the target zone unbound RNA sample;
depositing said signal-DNA in said flow channel;
allowing signal-DNA to hybridize to said target-RNA in the target zone, said signal-DNA and said capture-DNA being non-complimentary;
removing from the target zone unbound signal-DNA;
depositing said reporters in said flow channel;
allowing reporters to bind to the bound signal-DNA so that reporters associated with the target-RNA are maintained within the capture zone;
removing from the target zone unbound reporters; and
detecting reporters in the target zone to thereby determine whether target-RNA is present in the RNA sample.

60. The method according to any one of claims 57, 58, or 59 wherein said step of removing unbound reporters is performed by rotating the optical bio-disc.

61. The method according to either claim 57 or 58 wherein the presence of said target-DNA is determined by directing a beam of electromagnetic energy from a disc drive assembly toward said target zone and analyzing electromagnetic energy reflected from the disc.

62. The method according to claim 59 wherein the presence of said target-RNA is determined by directing a beam of electromagnetic energy from a disc drive assembly toward said target zone and analyzing electromagnetic energy reflected from the disc.

63. The method according to either claim 57 or 58 wherein the presence of said target-DNA is determined by directing a beam of electromagnetic energy from a disc drive assembly toward said target zone and analyzing electromagnetic energy transmitted through the disc.

64. The method according to claim 59 wherein the presence of said target-RNA is determined by directing a beam of electromagnetic energy from a disc drive assembly toward said target zone and analyzing electromagnetic energy transmitted through the disc.

65. The optical disc according to any one of claims 1, 11, 21, 26, 27, 28, 29, or 43, wherein the reactive group is an amino group.

66. The surface assembly of claim 6, wherein the reactive group is an amino group.

67. The method of any one of claims 31, 32, 33, or 34, wherein the reactive group is an amino group.

68. The method of any one of claims 38, 39, 40, or 41, wherein the reactive group is an amino group.

69. The method of any one of claims 50, 51, 52, or 53, wherein the reactive group is an amino group.
